(19) United States
(12) Patent Application Publication
(10) Pub. No.: US 2010/0196871 A1
(43) Pub. Date: Aug. 5, 2010

(54) APPARATUS AND METHODS FOR CULTURING AND/OR TRANSPORTING CELLULAR STRUCTURES

(76) Inventors: John R. Dodgson, London (GB); Malcolm T. Austen, Middlesex (GB); Bryan J.A. Miller, Middlesex (GB); Raymond A. Cattini, Hampshire (GB)

Correspondence Address: Goodwin Procter LLP;Attn: Patent Administrator, 135 Commonwealth Drive, Menlo Park, CA, 94025-1105, US

(21) Appl. No.: 12/596,954

(22) PCT Filed: Apr. 23, 2008
(86) PCT No.: PCT/GB08/01427

§ 371 Date: Mar. 17, 2010

(30) Foreign Application Priority Data
Apr. 23, 2007 (GB) 0707776.1

(51) Int. Cl. A01N 1/02; A01N 1/00; C12M 1/00; C12N 5/02

(52) U.S. Cl. 435/1.1; 435/284.1; 435/289.1; 435/307.1; 435/283.1; 435/383

(57) ABSTRACT

An apparatus for culturing and/or transporting embryos, oocytes or other cellular structures, comprising a housing (12) having a gas space (28) and a media space (18) for a liquid medium separated from one another by a barrier (16) having one or more gas permeable regions to allow gas diffusion from the gas space to the media space, and an essentially gas-tight gas closure means (14) adapted to restrict the passage of gas into the container from the exterior environment, and liquid closure means (14, 16) adapted to engage with the housing to form a liquid-tight seal for retaining liquid in the media space. The barrier preferably comprises an at least partly gas-permeable insert (16) that when inserted into the housing forms together with the housing a gas space (28) and a liquid media space (18) in gas communication with one another via diffusion through the insert. The insert may be at least partly porous, the pores of the insert comprising at least part of the gas space.

### TECHNICAL FIELD OF THE INVENTION

This invention relates to apparatus and methods for culturing cells, maturing oocytes and culturing embryos and other cellular structures in vitro, and means of transportation of cells, oocytes, embryos and other cellular structures.

### BACKGROUND TO THE INVENTION

Various apparatus and methods are known for maturing oocytes and culturing embryos in vitro. In standard practice these processes are achieved using conventional tools such as Petri dishes and well-plates with large wells, such as 4-well to 24-well plates, to contain the oocyte or embryo and maturation or culture media. The oocytes or embryos are usually cultured in an incubator in conditions of controlled temperature and gas environment. They may be cultured singly or in groups, and for oocytes in particular, may be cultured in the presence of other cells, such as cumulus cells. Maturation or culture is often done in microdrops of media in a Petri dish or well plate, the media covered by inert oil, the media being free to exchange gas with the environment in the incubator. Respiration of the embryo(s) or oocyte(s) is sustained by diffusion of oxygen through a relatively shallow depth (a few mm) of media and oil. The media is usually bicarbonate buffered and the pH is kept constant by means of equilibrium with CO2 in the incubator atmosphere. In some conventional maturation or culture procedures the volume of the media environment in which the oocyte or embryo is contained is important—there is evidence that maturation and culture is more successful if several oocytes or embryos are present together in a small volume of media. This auto/paracrine effect is thought to result from trace chemical substances produced by a first oocyte or embryo affecting the development of a second. Therefore small volumes of media per embryo or oocyte are used—typically 10-20 μl per embryo for bovine embryos, though this volume is smaller in some protocols.

Such systems are unsuitable for transport of embryos or oocytes, and in general practice these are transported, in a portable incubator, inside a sealed vial completely filled with media. This has the disadvantage that gas transport to the embryos or oocytes is from the media only, rather than from a gas atmosphere separated from the embryo(s) or oocyte(s) by a thin liquid layer. The increased diffusion limitation and relatively low solubility of oxygen in aqueous media may lead under some circumstances to development of a hypoxic environment around some or all of the embryos. There is a secondary effect of loss of gas from the media to the atmosphere through the walls of the vial if this has significant permeability. Also, a considerable volume of media is typically used—much larger than the typical volume per embryo used in microdrop culture—so negating the beneficial effects of group culture.

A further requirement is the ability to access certain embryos at a given time at the point of use (typically embryo transfer (ET)). An effective embryo transport apparatus should be usable away from conventional laboratory facilities, allowing a subset of the embryos to be removed while maintaining a controlled gas atmosphere for the unused embryos. This means that ideally a transport apparatus should have partitioned gas environments that can be opened selectively, or a means to replenish the common gas environment from a gas source; though the latter course will require a gas cylinder with consequent weight penalty and regulatory complication.

A further requirement is the ability for transport to be done using conventional shipping in which transport might be in any orientation, the transportable incubator in which the transport container is housed might be dropped or shocked, and shipping might take a significant part of the total embryo culture time, for example up to 72 hours in cases where delays are encountered.

The transport container should also be easy to use and of low cost, as it is preferably a single use, disposable item in common with most laboratory culture equipment. To this end, it is advantageous if the transport apparatus is designed to make use in its design or assembly of conventional laboratory components so far as is practical. The material of the container should be inert at incubation temperature (around 37 C) with respect to leaching of component substances into the media, to avoid the possibility of embryotoxicity. There is widespread doubt about the applicability of relatively low melting point polymers such as polyethylene, polypropylene and other materials such as are found in supplies and containers for cryopreservation, when operated at incubation temperatures for long periods (several hours or more).

A number of apparatus and methods have been proposed for transportation of embryos while maintaining improved culture conditions. None of these have addressed satisfactorily the above concerns.

Seidel et al., U.S. Pat. No. 7,094,527, disclose an embryo transportation apparatus and method comprising a 0.1-0.5 ml volume ET straw filled with media, oocytes and sperm (to achieve fertilisation in situ and then culture and transport the resulting embryos), heat sealed and enclosed within a secondary container or ‘incubation element’ that contains a desired gas atmosphere. Seidel et al. give very few details of the apparatus and the method of use. They do not discuss means to provide gas (oxygen, carbon dioxide) access to the media inside the straw, and the requirement for this is not discussed. By implication the source of controlled gas composition is diffusion through the walls of the straw and the media between the wall and embryos. For a single embryo or a sparse group of embryos resting on the sidewall of the straw oxygen diffusion through the wall will be sufficient to maintain the non-diffusion-limited respiration rate of the embryos (for bovine embryos, approximately 1.4E-14 mol.s-1, H. Shiku et al., Anal. Chem. 73(15) (2001) 3751-8). However, if a closely-spaced group of embryos sediments together, as will happen in particular at a bottom corner of the straw if the straw is transported close to vertically, and especially in a 5% O2 atmosphere as is typically used for culturing bovine embryos, oxygen supply by diffusion to the group of embryos will fall below the optimum value and hypoxic conditions may be established within or around the group. The fixed volume capacities of commercially available straws mean that for the ideal culture volume a set number of embryos should be placed in each straw: e.g., for 10 ul per embryo, 10 for a 0.1 ml straw and 50 for a 0.5 ml straw. If fewer embryos are desired to be shipped, then either the volume per embryo will be greater, or the straw can be partly-filled—but this creates the risk that the liquid column will break up through movement of the straw during transport, leaving embryos in unpredictable liquid volumes, or even dry on the side of the straw. Seidel et al. quote a figure of 10-15 embryos per 50 μl for fertilisation, which is proposed to be done inside the straw, so implying 20-30 presumptive zygotes in a 0.1 ml straw and 100-150 in a 0.5 ml straw, which gives and even greater risk of hypoxia if a substantial proportion of the zygotes are viable and they are closely grouped together at the base or at a corner.

An additional problem is that ET or cryopreservation straws are not designed for prolonged (many hours) contact with media at incubation temperatures: the internal surface area/volume ratio of the straw is high, and the material of the straw is not necessarily inert at incubation temperatures and so may leach trace compounds into the media that compromise embryo development. Embryo straws are intended for use in cryopreservation, at which temperatures the material will leach very slowly, if at all, and the embryos are not metabolically active while in contact with media that may have trace leached compounds within it. The need for the straw to be heat-sealable limits the material to a small group of compounds that have relatively low melting point and consequently have greater surface openness to diffusion of mobile contaminants than polymers with higher melting points.

In summary, the embodiment of Seidel et al. does not achieve the desired ends of a known, predictable gas supply, equivalent operation in any orientation during transport and a controlled, small volume for incubation.

Thouas et al., WO02/074900, disclose incubation inside a capillary open at the ends, in which one or two embryos are placed inside a glass capillary and are incubated preferably while the capillary is vertical, the embryo(s) resting on the liquid/gas meniscus. This provides good gas access to the embryos, and a small, controlled volume of media; the high aspect ratio of the media space means that in fact the effective volume experienced by the embryo(s) in terms of accumulation of auto/paracrine factors will probably be smaller than the total volume, which is in any case much smaller (around 1 ul) than is used in standard protocols. Establishing a 10 μl per embryo culture volume in the system of Thouas et al. would mean the capillary would have to have a much larger diameter and so have poorer retention of the media against movement, shock etc. The glass capillary system does not allow groups of embryos to be cultured inside the capillary away from the meniscus as the glass is impermeable and oxygen transport through the media from the ends of the capillary is too limited. Additionally the system is not operable in any orientation—even a single embryo will suffer limited oxygen supply if it is located in a glass capillary far from the gas supply at the open end.

Ranoux et al., US2006/0228794, disclose an embryo culture container for use primarily in human intravaginal embryo culture, comprising a gas-permeable inner vessel with a closure device for ‘selective access’, at least partially surrounded by a shell that defines a buffer chamber for a controlled gas atmosphere. During incubation in a controlled gas environment the buffer chamber is open to the surrounding environment (e.g. the vagina) via a gas-permeable seal region. When the container is removed from the controlled gas environment the gas path between the surrounding environment and the buffer chamber can be closed by a second closure mechanism associated with the shell. The vessel is adapted for culture of one or a few embryos, and comprises a large chamber, of volume not stated but large enough to admit a catheter or pipette, and a ‘microchamber’, a narrow region of the main chamber, at the bottom of the vessel where the embryos can sediment for inspection. The container of Ranoux et al. addresses some of the concerns for effective embryo transport, but does not achieve a small volume of media per embryo and is not suitable for transport of a group of more than two or three embryos, owing to the use of low-permeability plastic for the inner vessel (which needs to be of rigid material, so precluding use of a high-permeability elastomer), the small dimensions of the microchamber, and the possibility that a group of embryos would accumulate there during transport so potentially inducing hypoxic conditions. The design of the container, with a closure means formed as part of the inner vessel, is inherently difficult to adapt to a much smaller total media volume. It is also a complex and relatively costly design, not well suited to a single-use container for transport of non-human embryos for commercial purposes.

Campbell et al., US2002/0068358, have proposed an apparatus for embryo culture which is adapted for transportation, in which the embryo is retained in a well that has a supply of media and flow generating means to allow the media in the well to be replaced under remote or automatic control. The well is considerably larger than the embryo, so giving poor control of the media environment; there is no provision for oxygen supply to the embryo(s) except through flowing media past them, so precluding build up of beneficial auto/paracrine factors around the embryo(s). Additionally, the design is highly asymmetric, with access to the well through a long tube of significant size and internal volume, and so is unsuitable for operation upside down.

It is an object of the present invention to address these and other difficulties in the design and operation of embryo culture and transport devices of the prior art.

In the description that follows reference will be made to culture and transportation of embryos as an example of the function of apparatus and description of the method. Many of the processes can also be applied to maturation and transportation of oocytes and culturing and transportation of cells or other cellular entities and it will be apparent to those skilled in the art how this application can be made, with appropriately chosen dimensions for the different size scales of embryos, oocytes and cells. Therefore the terms maturation and culturing, and oocytes and embryos and cells, are used interchangeably in the following and where convenient referred to collectively as ‘objects’. Where specific features of the invention apply to maturation of oocytes, or to culturing of embryos, this will be noted.

### SUMMARY OF THE INVENTION

According to a first aspect, the invention provides a container for culturing and transporting embryos, oocytes and other cellular structures as specified in claims 1 to 20.

According to a second aspect of the invention there is provided an insert for a container for culturing and transporting embryos, oocytes and other cellular structures as specified in claim 21.

According to a third aspect of the present invention, there is provided an apparatus for transporting embryos, oocytes and other cellular structures as specified in claim 22.

According to a fourth aspect of the present invention, there is provided a method of culturing embryos or oocytes as specified in claims 23 to 26.

A preferred embodiment of the present invention is provided by a container comprising a housing, having an at least partly gas-permeable insert that is an interference fit within the housing, the housing and the insert defining at least one gas space and at least one liquid media space in gas communication with one another by diffusion through the insert, and a separable, essentially gas-tight closure means that cooperates with the housing to restrict the passage of gas into the container from the outside environment.

In another preferred embodiment, the container comprises a housing, an insert having an inner face oriented towards the inside of the housing and an outer face, the insert being mounted within the housing so defining a gas space between the inner face of the insert and the housing, the insert comprising a recess open to the outer face, the recess adapted to be a space for liquid media. The insert is preferably formed from a gas-permeable polymer and has one or more walls thin enough (either in whole or part) to give good gas exchange (oxygen and carbon dioxide) with the atmosphere in the gas space. The container further comprises a cap that closes and seals both the gas space and the media space.

Optionally the insert when in place in the housing substantially closes the gas space from the external atmosphere so that without the cap fitted gas exchange between the gas space and the atmosphere is primarily by diffusion through the material of the insert.

Optionally the insert comprises a vent channel or one or more regions of open-pore material so that a gas-phase path exists between the gas space and the atmosphere, this path being closed along with the media space when the cap is fitted.

In an alternative embodiment the container comprises a housing, an insert that is mounted within the housing defining below it a media space, the insert and housing defining a gas space that is open to the atmosphere but which can be closed by a cap.

In a further preferred embodiment the container comprises a housing having a number of wells, and an insert having a number of projections that are adapted to fit into the wells, so defining below them a gas space in each well, each projection comprising a recess open to the outer face of the insert, the recess adapted to be a space for liquid media. The insert is preferably formed from a gas-permeable polymer and has one or more walls thin enough (either in whole or part) to give good gas exchange (oxygen and carbon dioxide) with the atmosphere in the gas space. The container further comprises a cap means that seals both the gas spaces and the media spaces.

Optionally the insert substantially closes the gas space from the external atmosphere so that without the cap fitted gas exchange between the gas space and the atmosphere is by diffusion through the material of the insert.

Optionally the insert comprises a vent channel or one or more regions of open-pore material so that a gas-phase path exists between the gas space and the atmosphere, this path being closed along with the media space when the cap is fitted.

Optionally a further secondary lid or container is provided that encloses the container so establishing a controlled gas atmosphere on the top side of the container, the bottom side or both.

In an alternative embodiment the container comprises a housing having a number of wells, and an insert having a number of projections that are adapted to fit into the wells, so defining below them a media space in each well, each projection comprising a recess open to the outer face of the insert, the recess adapted to be a channel for diffusion of gas to the media space. The insert is preferably formed from a gas-permeable polymer and has one or more walls thin enough (either in whole or part) to give good gas exchange (oxygen and carbon dioxide) of the objects in the media with the atmosphere in the gas space. The container further comprises a cap that seals both the gas spaces and the media spaces.

### BRIEF DESCRIPTION OF THE FIGURES

FIG. 1a shows a first view of a first embodiment of an apparatus according to the invention

FIG. 1b shows a second view of a first embodiment of an apparatus according to the invention

FIG. 1c shows a third view of a first embodiment of an apparatus according to the invention

FIG. 2 shows a second embodiment of an apparatus according to the invention

FIG. 3a shows a first view of a third embodiment of an apparatus according to the invention

FIG. 3b shows a second view of third embodiment of an apparatus according to the invention

FIG. 4 shows a fourth embodiment of an apparatus according to the invention

FIG. 5 shows a fifth embodiment of an apparatus according to the invention

FIG. 6 shows a sixth embodiment of an apparatus according to the invention

FIG. 7a shows a seventh embodiment of an apparatus according to the invention

FIG. 7b shows an eighth embodiment of an apparatus according to the invention

FIG. 8 shows a ninth embodiment of an apparatus according to the invention

FIG. 9 shows a tenth embodiment of an apparatus according to the invention

FIG. 10a shows an eleventh embodiment of an apparatus according to the invention

FIG. 10b shows a detail of an eleventh embodiment of an apparatus according to the invention

FIG. 10c shows a detail of an eleventh embodiment of an apparatus according to the invention

FIG. 11 shows a twelfth embodiment of an apparatus according to the invention

FIG. 12 shows a thirteenth embodiment of an apparatus according to the invention

FIG. 13a shows a fourteenth embodiment of an apparatus according to the invention.

FIG. 13b shows a fifteenth embodiment of an apparatus according to the invention.

FIG. 14 shows a sixteenth embodiment of an apparatus according to the invention.

FIG. 15 shows a seventeenth embodiment of an apparatus according to the invention.

FIG. 16 shows an eighteenth embodiment of an apparatus according to the invention.

FIG. 17 shows a further embodiment of an apparatus according to the invention.

### DETAILED DESCRIPTION OF EMBODIMENTS OF THE INVENTION

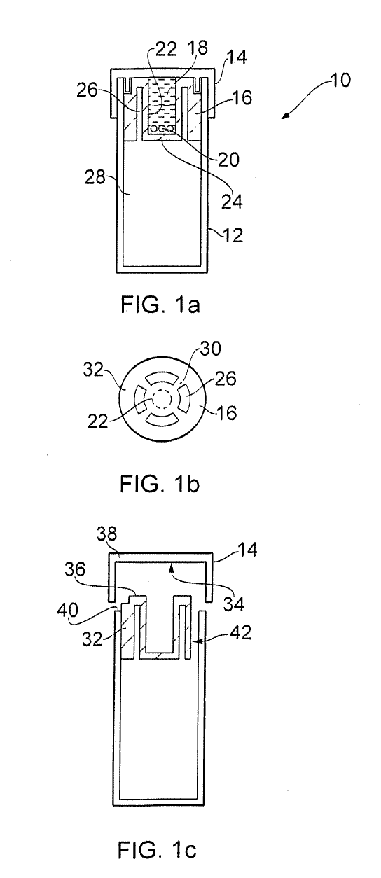

FIG. 1 shows a first embodiment 10 of a container according to the invention, comprising a housing 12, a cap 14 and an insert 16. The cap 14 fits to the housing 12 so as to form a substantially gas-tight seal, in preferred embodiments by means of a screw thread on the exterior of the housing (not shown). The insert 16 comprises a media space 18 adapted to contain media and embryos 20. The media space 18 is bounded by walls 22 and base 24, and closed by the cap 14 when this is fully fitted to the housing. The insert and housing together define a gas space 28 within the housing. In the embodiment shown in FIG. 1a the insert comprises one or more gas channels 26 that pass into the body of the insert, so defining one or more thinner sections of the wall 22. The gas channels 26 allow rapid gas phase diffusion between the gas space 28 and the wall 22 of the media space.

FIG. 1b shows the bottom view of the insert in FIG. 1a. Four gas channels 26 are shown, separated by bridges 30, which act to connect the wall 22 of the media space with the outer ring 32 of the insert. It is understood that any practical number and size of gas channels may be provided.

FIG. 1c show the configuration of the housing 12, cap 14 and insert 16 as the container 10 is assembled. The insert is sized so that it is a friction fit to the container, so on first insertion into the container it is retained with the outer part projecting from the top of the housing. The media space 18 can now be filled with media and embryo(s). To seal the container the cap is pressed down onto the top of the insert and either pushed or screwed (depending on the cap fitting method) down onto the housing. The inner face 34 of the cap bears on the sealing face 36 of the insert, and as the cap moves downwards onto the housing the insert is forced down into the housing against a reaction force from friction between the outer ring 32 and the inner wall of the housing. When the cap is fully screwed into place, the seal face 38 of the cap seals onto the top edge 40 of the housing, so providing a substantially gas-tight seal; the inner face 34 of the cap is pressed hard up against the seal face 36 of the insert, and the cap simultaneously closes both the media space 18 and gas access to and from the gas space 28. In some embodiments a vent channel 42 is provided that acts to vent gas from the gas space 28 when the insert 16 is forced down by the cap as this is fitted. If present, the vent channel is preferably of small cross section to limit the degree of gas exchange between the gas space and the external atmosphere via the channel.

While the invention is not limited by having the features shown in FIGS. 1a, 1b and 1c, these features are advantageous in this embodiment. The gas space 28 can be filled with a gas mixture appropriate for culture of embryos in the media. The mixture will depend on the species but the container of the invention is particularly suited for species in which low O2 content gas is typically used, such as bovine embryos: the walls 22 and base 24 of the media space are preferably thin to allow ready diffusion of O2 and CO2 across them. The gas channels 26 reach up towards the regions of the insert close to the cap so allowing good gas access to areas of wall close to the cap. This allows good gas supply to the embryos if the container is held or transported upside down, so that the embryos rest against the cap. The design allows a degree of radial compressibility that is set by the thickness and o.d, of the outer ring 32, with a contribution of some resistance to compression from the bridges 30, and a lesser degree of axial compressibility. The insert can then be designed to give good friction against the housing when the container is being closed, resulting in a small degree of axial compression of the insert which acts to keep the media space closed by the lid. Friction is assisted if the housing 12 is tapered, but this an optional feature which is not essential in practice.

The housing 12 and cap 14 can be made from a range of rigid materials, such as plastics, glass, metal or the like. Advantageously the housing and cap are a mass-produced pair, such as a standard laboratory vial plus cap. The insert 16 is then designed and made to fit the pre-existing vial. Appropriate vials are for example Bibby Sterilin Bijou 7 ml (Barloworld Scientific Inc.), in polystyrene or glass. The insert is preferably at least partly resilient—for example a moulded elastomeric component, or a component comprising a rigid core with an elastomeric outer component to give a close friction fit against the wall of the housing. In some embodiments the housing material is impermeable to gas, e.g. glass. This allows the gas space to be filled with a chosen gas composition (e.g. 5% oxygen, 5% carbon dioxide, nitrogen) and to retain the composition while in a different atmosphere, e.g. air. In other embodiments the housing may be permeable to gas, allowing the gas space to exchange gas with its surroundings. If the housing is permeable, the container may be used in air provided the permeability is low enough that the gas atmosphere is retained for the desired culture period. Example 1 describes a preferred design for this embodiment.

The insert material is preferably gas-permeable, and in preferred embodiments is sufficiently permeable that, together with the design of the wall and base thickness and the dimensions of the gas channels 26, the impedance to diffusion of oxygen through the insert is not limiting on oxygen supply to embryos within the gas space. In preferred embodiments the insert is moulded from PDMS, for reasons of high gas permeability, ease of moulding and sterilisation, and lack of embryo toxicity. Suitable PDMS compounds are Silastic S and Sylgard 184 (both from Dow Corning), but others will be suitable also (subject to embryo toxicity checks).

In this embodiment, and in all other embodiments of the invention, the material of the insert may be coated with a surface coating, or the surface chemically modified, by any means known in the art in order to modify the surface properties of the insert, in particular in the region contacting the media space. This might be done to modify for example the absorption or adsorption properties of PDMS, the gas permeability or the ease with which embryos, oocytes or cells adhere to the surface.

In use the gas atmosphere may be introduced into the gas space 28 either by actively flushing the gas space with a gas stream before placing the insert in the housing, or the housing with the insert fitted in place (preferably partially in place as shown in FIG. 1c) may be placed in a gas atmosphere, for example in a conventional incubator, and the contents of the gas space allowed to equilibrate with the atmosphere by diffusion through the material of the insert. As the insert is preferably made from a material with high gas diffusion coefficient, and the walls and base of the media space 18 are thin, equilibration is fast enough for this to be an effective mode of use. The equilibration times needed depend on the material and dimensions of the insert and the dimensions of the housing. Example 1 illustrates the typical gas exchange time for a preferred design.

The insert might also be incubated with water or media of the type to be used in place in the media space, in order that any adsorption or absorption processes associated with the properties of the insert material are completed before the culture media is dispensed.

When the gas space has reached equilibrium the container can be removed from the incubator and loaded with media and embryo(s). In preferred embodiments the equilibration time is sufficiently long that this can be done in the laboratory without significant change in the composition of the gas in the gas space. The cap 14 is then fitted, forcing the insert 18 down into the housing and sealing both the media space and gas access (via diffusion through the insert material) to the gas space.

The container 10 is sized to contain a media space of volume chosen according to the preferred media volume per embryo and number of embryos desired to be cultured. Preferred embodiments have media space volumes between 1 ul and 5 ml, more preferred embodiments volumes between 10 μl and 1 ml and most preferred embodiments between 50 μl and 500 μl. The container may house embryos at any preferred ratio of media volume per embryo. Preferred embodiments contain embryos at ratios between 1 embryo per 0.5 ul and 1 embryo per 100 μl, more preferred embodiments ratios between 1 embryo per 1 ul and 1 embryo per 100 μl, and most preferred embodiments ratios between 1 embryo per 2 ul and 1 embryo per 10 μl. It is a particular advantage of the invention over the containers and culture apparatus of the prior art that small volumes of media per embryo can be used in a configuration that is easy to access and can be shipped in a robust manner, with substantially all the oxygen requirement of the embryos met whatever the orientation of the container.

The housing 12 is sized to contain sufficient gas to sustain the metabolism of the embryos in the container and to allow for leakage from the gas space to the surrounding atmosphere where that differs from the gas in the gas space. Such leakage will occur through the walls of the housing, if that is not made from an impermeable material such as glass; through the seal between the cap and the housing, and through the material of the cap. For a housing made from polystyrene preferred volumes of the gas space 28 are between 1 ml and 20 ml. Volumes less than 1 ml will give relatively little time before the composition in the gas space changes as a result of diffusion through the housing wall; volumes greater than 20 ml are unnecessary for preferred numbers of embryos in the media space and typical times in transit (see example 1 below). However, the volume in practice can be set by choice of practically available and/or easily handlable housings and caps and any volume of gas space 28 is within the compass of the invention.

The dimensions of the gas channels 26 are set by the amount of oxygen required by the embryos in the media space, in the condition that the container is oriented other than in the standard base-down way, in which case the embryos will have sedimented onto a wall 22 of the media space or onto the surface 34 of the cap. In this case they will draw oxygen by diffusion through the material of the wall or, if they are on the cap surface 34, by hemispherical diffusion through the media, ultimately from the wall 22. In this latter condition the availability of a gas-phase oxygen source close by avoids limitation of oxygen supply by diffusion, and in preferred embodiments the channels 26 approach close to the sealing face 36 of the insert in order to deliver gas-phase oxygen in close proximity to the cap surface 34. The dimensions of the gas channels 26 are unimportant in terms of the rate of diffusion of oxygen along them—diffusion in the gas phase is so much faster than in the media or insert material that effectively no diffusion limitation will exist along the length of the channels. The dimensions of channels 26 are therefore set effectively by practical moulding considerations—they are formed, for example, by a narrow upstanding feature on the mould and so are made as large as is practical to minimise the effect of wear and damage on the mould tool.

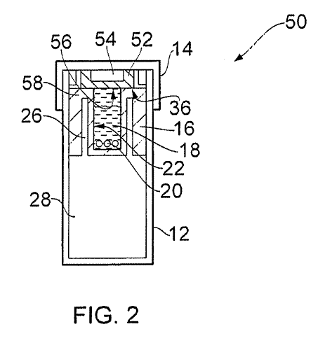

FIG. 2 shows a further embodiment 50 of the invention, in which similar parts to those in FIGS. 1a-c are shown with the same numerals. The container 50 is similar to container 10 in FIGS. 1a-c, but has an additional gas-permeable lid component 52 mounted between the lid face 34 and the inert 16. The lid component 52 may be attached to the lid, or placed over the insert 16 when the lid is to be attached, and held in place by the lid while the lid is screwed or pushed down. The lid component 52 acts to ensure access of gas to the end of the media space closest to the lid. This can be important if the container is designed to hold a number of embryos, and needs to ensure no oxygen supply limitation when the container is held upside down or partially upside down. Embryos 20 will then rest either on the surface 56 of the lid component 52 or at the corner between surface 56 and wall 22. Gas access is via the channels 26, through the permeable material in region 58 to the lid component 52. The lid component might comprise a gas space 54 for ready gas diffusion across the diameter of the lid component, so ensuring substantially uniform oxygen supply at all parts of the surface 56.

The insert 16 for container 50 is designed in a similar way to that for container 10, but is now designed to be displaced further into the housing when the lid and lid component are forced down by fitting of the lid. As in container 10, fitting the lid seals both the media space 18 and the gas space 28 in one action.

In an alternative embodiment, the lid component has a plug-like form so that it fits inside, and seals against, the upper portion of the wall of the media space 18. In this case the lid component might be separate from the lid, and adapted to be fitted to the insert 16 before the cap 14 is fitted, lid component 52 then acting as a stopper to seal media space 18 and cap 14 when fitted acting to seal both the top of the media space and the gas space 28 from the outside atmosphere.

In preferred embodiments the lid component 52 is moulded in PDMS, and where lid component 52 is adapted to fit inside insert 16 the lid component and insert are preferably of different grades of hardness of PDMS. Example 2 shows the effect of this embodiment on availability of oxygen to embryos resting on the surface 56 of the lid component.

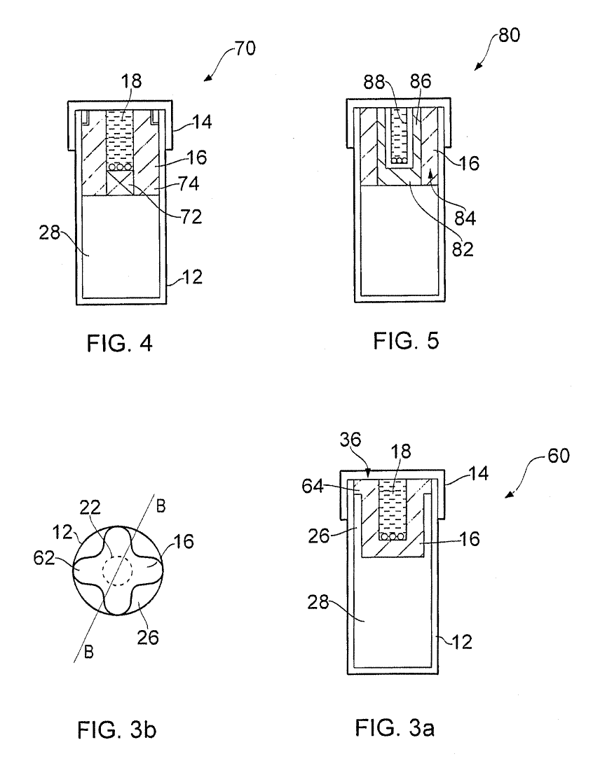

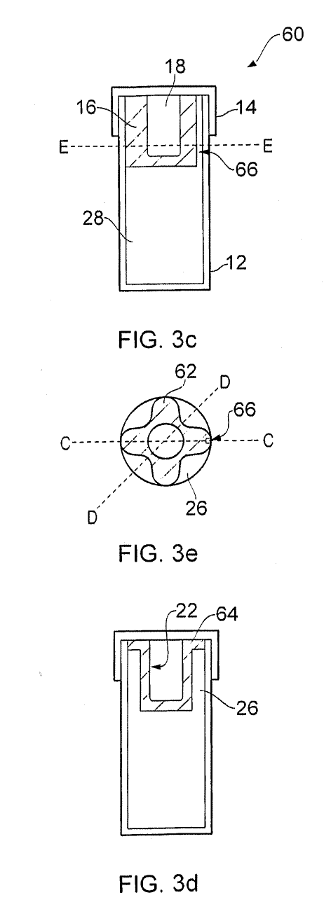

FIG. 3a and FIG. 3b show a further embodiment of the invention. Container 60 comprises a housing 12, cap 14 and insert 16 as in previous embodiments, but in this embodiment the gas channels 26 are formed as depressions in the external surface of the insert body, rather than as recesses within it. This has the advantage that the gas channels can be more readily moulded, and the moulded insert will detach more readily from the mould, than in the previous embodiments in which the channels are formed as blind holes. This is particularly advantageous in embodiments where the media space 18 is small, for example to hold few embryos or an individual embryo, and consequently the insert itself is small, and the parts of the mould tool needed to form blind hole gas channels would be small and easily damaged. In some embodiments the channels 26 are formed over the whole length of the insert, rather than stopping short of the sealing face 36. In this case the gas channels 26 form complete gas phase pathways between the gas space 28 and the lower face of the cap 14 when this is in place. The cap 14 acts as before to seal gas access between the gas space and the external atmosphere. In such embodiments, when the container is open the gas path through channels 26 allows relatively rapid gas exchange by diffusion between the gas space 28 and the exterior and so the composition of the gas in the gas space will change appreciably unless the media and embryos are placed in the media space and the cap closed quickly. In preferred embodiments a rim 64 is provided around the periphery of the insert, shown in FIG. 3a as adjacent to the sealing face 36, but optionally at another position along the length of the channels 26. The rim 64 acts to close or constrict the channels 26 and so reduce diffusion of gas between the gas space and the surrounding atmosphere. The rim 64 is in contact with the housing 12, and may close the channels 26 completely, substantially or partially, to achieve the desired degree of limitation or restriction.

FIG. 3b shows the base of the insert 16, with B-B showing the position of the cross section in FIG. 3a. The insert may have any number of channels 26 and projections 62 that bear on the wall(s) of the housing 12 when assembled. The channels 26 are shown in FIG. 3b as being relatively large, with a large radius profile that allows the insert and container to be made small, in turn allowing a small media space and small volume of media per embryo. The important features in the design are that the projections 62 give sufficient friction to provide a sealing counter-force when the cap is fixed in place; the walls 22 in their thinner region(s) provide sufficiently low resistance to gas diffusion to allow oxygen influx to sustain respiration.

FIGS. 3c-e show cross sections of a further embodiment of the invention, with similar parts to the embodiment in FIGS. 3a, b. FIG. 3c shows a cross section at C-C on FIG. 3e, FIG. 3d shows a cross-section at D-D on FIG. 3e and FIG. 3e shows a cross-section at E-E in FIG. 3c. The container 60 comprises a housing 12, cap 14 and insert 16 as before, the insert having walls 22 of the media space 18 with variable thickness so defining gas channels 26, in a similar manner as in the embodiment in FIGS. 3a and 3b. The gas channels 26 are substantially closed at one end by the rim or flange 64. A gas passage 66 is provided which, before cap 14 is fitted, leads from the gas space 28 to the external atmosphere. Gas passage 66 acts to vent gas from the gas space when the insert 66 is forced down into the housing by the cap when this is first fitted. In preferred embodiments the gas passage 66 is provided within the region of contact between a projection 62 of the insert and the wall of the housing, so allowing passage 66 to be long and narrow—this slows exchange of gas between the gas space and the external atmosphere by diffusion when the vial is uncapped. The embodiment in FIGS. 3c-e achieves the advantage of being easy to mould, with venting to prevent build up of pressure in the gas space when the insert is forced home, while maintaining slow enough inter-diffusion of gas through the vent gas passage 66 that the container can be handled in the laboratory outside a controlled gas environment for a practically useful time. With typical dimensions of the container (see example 3) the time constant for gas exchange before the cap is fitted can be around 1 hour, with a consequent time for a 5% carbon dioxide atmosphere to fall to 4.8% in laboratory air of around 2 minutes.

FIG. 4 shows a further embodiment of the invention. The container 70 comprises a housing 12, cap 14 and insert 16 as before. The insert 16 comprises an outer resilient component 74, formed for example from an elastomer such as PDMS, surrounding an inner porous component 72. The porous component 72 may be a closed-pore material with high gas permeability, but is preferably formed from an inert hydrophobic open-pore polymeric filter material such as porous polypropylene (e.g. VYON™, supplied by Porvair Ltd., Wrexham, UK) or porous PTFE (for example as available from Porex Inc., Fairburn, Ga., USA). The porous component 72 provides improved gas access to the base of the media space 18. In a preferred embodiment, a hydrophobic open-pore porous component 72 provides ready gas flow through the insert 16 into the media space 18, which avoids build up of pressure in the gas space 2S when the insert is first pushed into the housing, and provides good gas access to media and embryos in the media space when this is filled, while media is prevented from entering the pores.

FIG. 5 shows a further embodiment in which the container 80 comprises an insert 16 formed from an outer component 84 and an inner porous component 82 in which the media space 18 is formed. The porous component now wholly or substantially forms the walls of the media space 18 (except for the opening that is closed by the cap 14), and so allows good gas access through the porous structure to all regions of the wall of the media space. In preferred embodiments the porous component 82 is formed from an open pore hydrophobic porous material as in the description for the embodiment shown in FIG. 4. The embodiment 80 is advantageous in cases where contact between the media and a polymer such as PDMS, which might adsorb or absorb potentially significant quantities of components from the media, is not desired. Porous hydrophobic filter materials such as VYON™ are essentially inert, while allowing good gas permeability. The media space 18 might be lined by an inner layer 88 of material with different properties from those of the bulk porous material, for example of lower porosity to resist media ingress which might result from slight pressurisation while fitting the cap, or which is hydrophilic to provide a media-containing surface on which the embryos might rest. VYON™ material can be supplied in two-layer configurations and the insert component 16 can be cast from that material to dimensions to fit suitable commercially available housings 12, such as sample vials.

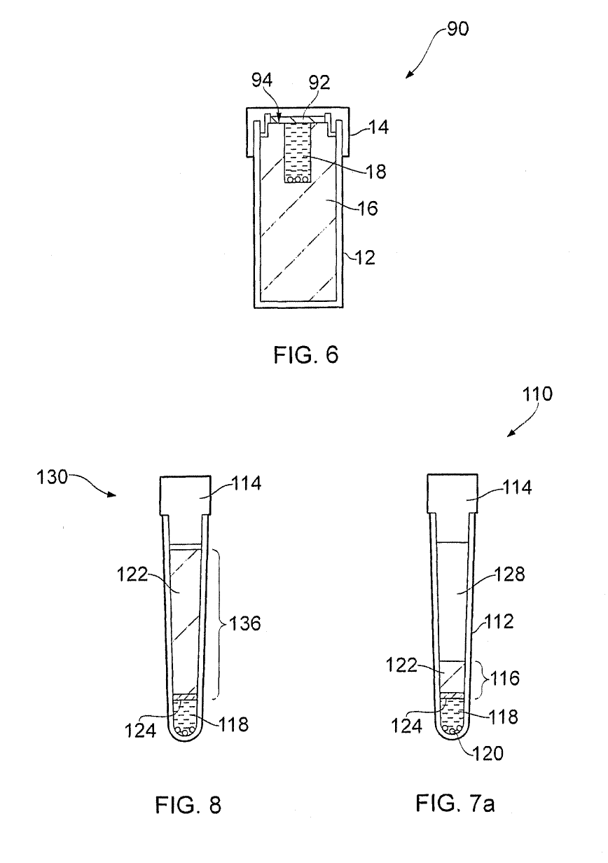

FIG. 6 shows a further embodiment of the invention. The container 90 comprises a housing 12, cap 14 and insert 16. The insert 16 comprises a porous material chosen to have high enough gas permeability that the gas in the pores can be exchanged by diffusion with a controlled gas atmosphere. The gas space is now located at least partially within the pores of the insert. The porous material might have closed pores, the permeability of gas through the walls of the pores being high enough to allow ready gas exchange between the bulk of the material and the exterior. In preferred embodiments the porous material is an open pore hydrophobic material such as VYON™, which allows ready exchange of gas between the bulk and the exterior, while preventing aqueous media from entering the pores. The media space 18 is preferably formed within the porous material, so providing gas access substantially all round the media space, and might have an inner wall formed from or coated with a second material as described for the embodiment shown in FIG. 5. The media space is sealed by the cap 14; if the porous media is insufficiently compressible to form a good seal against the inside face of the cap, a sealing component 92 is provided within the cap. The sealing component 92 might be attached to the cap or placed individually over the insert before the cap is fitted. Optionally the sealing component 92 might be in plug form, so that it is a push-fit into the media space 18, optionally being pushed into its final location by the action of fitting the cap. In the embodiment shown in FIG. 6 the insert reaches the base of the housing 12, so providing reaction force from the base of the housing against the action of sealing with the cap. This means that in some embodiments the upper face 94 of the insert may lie below the rim of the housing 12, the sealing component 92 extending from the inner surface of the lid down into the body of the housing 12. The insert 16 might occupy substantially all of the housing volume outside the media space, or may have voids that will contribute to the effective gas space.

In use the container 90 is pre-gassed by leaving it with the cap removed or only partially closed in a controlled gas atmosphere. Gas diffuses from the atmosphere into the pores of the insert, which act as a gas reservoir when the cap is closed. This embodiment has an operational advantage in certain circumstances in that the porous insert 16 might be gassed separately from the vial, being taken from the gas atmosphere and placed in the vial just before use. In that way the gas atmosphere is carried into the container within the pores of the insert; the insert physically displaces the air from the housing when it is inserted. The pore density and permeability are chosen so that the insert stores sufficient gas (determined by the pore volume) while having a suitable time constant for in-gassing in the incubator and slow enough out-gassing while handling in the environment that the gas atmosphere established inside the container is close enough to that desired.

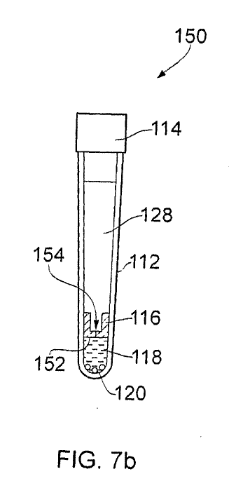

FIG. 7a shows a further embodiment of the invention. Container 110 comprises a housing 112, a cap 114 and an insert 116. In use the insert 116 is positioned inside the housing to define a media space 118 between the insert and the closed end of the housing and a gas space 128 between the insert and the end of the housing closed by the cap. The insert is a close fit to the housing so substantially preventing media from passing the insert to reach the gas space. In preferred embodiments the insert comprises an open cell porous hydrophobic material 122, which allows gas to pass through it but substantially prevents media from entering the pores. In preferred embodiments the housing 112 is tapered from open end to closed end to allow easy movement of the insert to its desired position. In preferred embodiments the insert comprises compliant material, so dimensioned that in its desired position it fits closely to the wall of the housing. The housing 112 may be formed from a range of materials, the choice being guided by gas permeability as before. As the embryo(s) is/are in direct contact with the material of the tube embryo toxicity also needs to be taken into account. A suitable housing is the Scimart 2.5 ml polystyrene sample vials, product code SAR-55483 and push fit cap SAR-65782. Suitable material for the insert is VYON™, as previously described.

In use, a known volume of media and embryo(s) are pipetted into the base of the housing 112; the insert is then fitted and moved down the housing until it has displaced substantially all the air between its bottom surface and the top surface of the media; in preferred methods of use the insert will be in contact with the media with no, or only small, air bubbles at the interface. The gas space 128 is then flushed with gas of the desired composition and the cap 114 fitted. The dimensions of the housing and insert are chosen so that the insert fits tightly to the housing at the desired position, i.e. when it is in contact with the media. In order to minimise disturbance to the gas concentration in the media the porous material of the insert may be pre-equilibrated with the chosen gas composition prior to fitting.

An optional additional layer 124 may be provided on the surface of the insert adjacent to the media space as described above for the embodiment shown in FIG. 5. Here the layer 124 may act also to assist sealing of the media space against flow of media past the insert, in which case the layer 124 is preferably formed from a gas-permeable elastomer such as PDMS. When this layer is present, the insert is preferably adapted so that the layer 124 may flex on insertion of the insert into the housing, so allowing gas within the housing to be displaced past the insert, the layer 124 regaining its shape when the insert is in position and so sealing against flow of media. This can be achieved for example by chamfering at least a portion of the surface of the porous component 122 adjacent to the layer 124, so providing a region into which a portion of the layer 124 can be displaced. Alternatively the layer 124 may be formed from a hydrophilic layer so providing a media-suffused region into which the embryos come into contact if the container is operated upside down.

FIG. 7b shows a further embodiment in which container 150 comprises similar parts to the embodiment 110 in FIG. 7a. The insert 116 is now formed from a material that allows a needle or tube to penetrate the insert, allowing media and objects to be dispensed into the media space 118 through a passage in the insert. This allows the insert to be mounted in the housing 112 before the media and objects are dispensed. In a preferred embodiment the insert comprises a membrane 152 in contact with the media space, which is preferably a thin area of material adapted to be punctured by a needle or pipette, in the manner of a septum. The membrane then re-closes once the needle or pipette has been withdrawn, sealing the media space from the gas space 128. In an alternative embodiment, the insert is provided with an opening 154 such as a slit, for example within the membrane 152, that opens when pressed by a needle or pipette to allow easy access through the insert, and re-closes when the needle or pipette is withdrawn.

In use the container may be provided as a set of a housing 112, insert 116 and cap 114, the insert being placed in the housing before use; or the insert may be pre-fitted into the housing so as to define the desired media space volume. The media space is then filled by means of the needle or pipette. The slit or opening is preferably sized so that air is displaced from the media space around the needle or pipette while media flows in, leaving no, or only small, bubbles in the media space. The gas space 128 is then gassed with the desired composition and the cap fitted.

The container might also be used as described for the embodiment in FIG. 7a, in which the media plus object(s) is dispensed into the housing first and the insert is then fitted. In this case the opening or slit 154 is advantageous, to serve to vent air from the below the insert 116 as this is pushed down into the housing.

In a preferred embodiment the insert is moulded from a gas-permeable elastomer such as PDMS, which can be formed to have good puncture/re-seal properties as exploited in septa. It will be apparent that the housing can be of different shape, for example a screw-top vial as in FIG. 1a, and that while the housing is advantageously tapered, this is not essential.

While the inserts 116 in FIGS. 7a and 7b are shown as being essentially disc-shaped and located in use in the lower part of the housing 112, the inserts might have one or more handles or projections to assist them to be placed in the housing. Such handles or projections might extend towards the mouth of the housing, and in preferred embodiments be so sized as to aid correct positioning of the insert in the housing.

FIG. 8 shows a further embodiment in which container 130 comprises similar parts to the embodiment 110 in FIG. 7a, except that the porous component 122 of the insert 136 now occupies most or substantially all of the housing 112 between the media space and the cap. In this embodiment the pores within the insert may form the main part of the gas space. In use the insert 136 may equilibrated with the chosen gas composition before being inserted into the container. Media and embryos are pipetted into the housing as before and the insert fitted. The insert is now the means of introducing the chosen gas composition into the container. This embodiment has the advantage that the insert 136 can be filled with gas by being left inside an incubator for an equilibration time, and no flushing of the container with gas is needed once the media and embryos have been loaded. The insert 136 is designed so that gas within the pores exchanges with the outside atmosphere sufficiently slowly that the insert can be handled outside the incubator while substantially retaining the desired gas composition. An optional surface layer 124 is again shown.

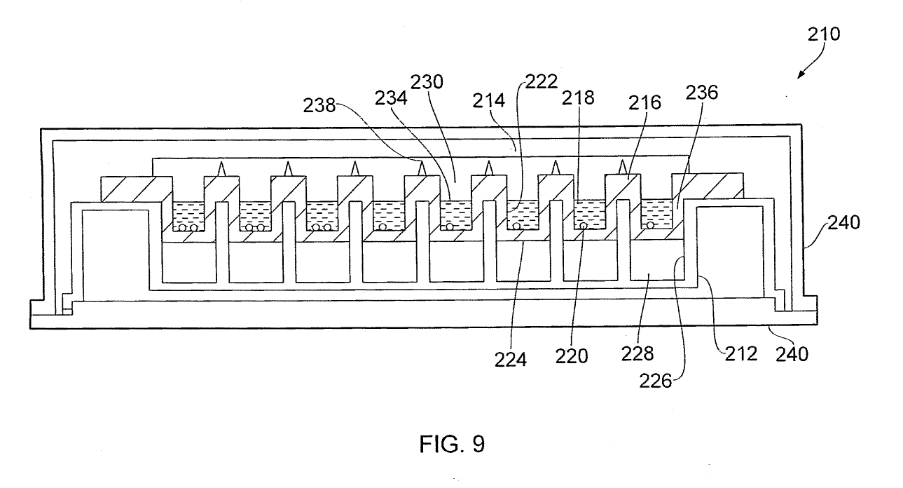

FIG. 9 shows a further embodiment of the invention that allows multiple single embryos or small groups to be cultured in controlled conditions in smaller volumes than are practical in prior art apparatus, while maintaining their separate identity. The container 210 comprises an assemblage incorporating a housing 212 that comprises one or more wells 226, for example in microtitre plate format. In preferred embodiments housing 212 is a conventional microtitre plate or microtitre strip, or assembly of strips, of appropriate dimensions (number of wells, well size and shape) to hold the desired number of embryos 220 in the chosen volume of media. The container further comprises an insert 216 provided with one or more well-closing projections 236 each adapted to fit closely a well in the housing 212, defining within one or more of the wells of the housing a gas space 228, and comprising one or more media spaces 218. In preferred embodiments each well 226 of the housing 212 is fitted by a projection 236, leading to a multiplicity of closed gas spaces in diffusive communication with the media spaces 218, distributed across the housing 212. The media spaces 218 are each closed by a cap means 214 comprising stopper projections 230. The cap means might be formed as a continuous sheet-like component that lies over the insert 216, the stopper projections being inserted into the media spaces 218 by pressing the two components together. The cap means might have notches or articulations 238 that allow the cap means a degree of flexibility while being applied or removed. Alternatively the cap means might comprise a number of subcomponents joined together for example by a thin laminate, so allowing them to be applied jointly and removed separately, for example in strips, allowing a subset of the media spaces 218 to be opened at any one time. In some embodiments the housing 212, insert 216 and cap means 214 are contained within an outer container 240 to further control the gas atmosphere around the container.

In use the insert 216 is mounted on the housing 212 and the projections 236 forced home into the wells in the housing. The assembly can now be incubated in the desired gas atmosphere for the time needed for the gas to exchange with air in the gas spaces 228. The insert may also be incubated with media in the media spaces to condition the insert well material, for example through adsorption of media components onto/into the walls. This is particularly relevant if the insert 216 is formed from an elastomer such as PDMS, some grades of which have an affinity for water sufficiently high to deplete media content from a small media volume. Fresh media and embryo(s) are then dispensed into the wells, and the cap means 214 fitted. The closed container will exchange gas with the external atmosphere through the material of the housing 12, the insert 216 and cap means 214. These materials may be chosen to give slow enough gas exchange—particularly loss of carbon dioxide—to be acceptable in use. If necessary a secondary container 240 may be provided to assist control of gas exchange.

The apparatus of FIG. 9 can have a range of dimensions, with typical values being as follows. The same kind of estimate as is made in example 1 for the embodiment in FIG. 1 can be made for the other embodiments described, including the multiple-well embodiments in FIGS. 9-11. In a preferred embodiment the housing 212 is a standard 96-well plate; each gas space has volume around 200 μl, which will maintain 5 embryos for 72 hr with a fall in oxygen concentration from 5% to 4.8% over that time, assuming the container is isolated from the surrounding atmosphere. In preferred embodiments the media space 218 may have a volume in the range 10-150 μl, a particularly preferred volume being around 50 suitable for 5 embryos at 10 μl per embryo. The material of the insert is preferably a high permeability polymer such as PDMS, which provides gas access to the whole of the well by diffusion from the gas space, through the walls parallel to the media space, and into the media space throughout its depth. The stopper means 214 is preferably formed from a more rigid material than the insert, for example from a rigid embryo-compatible polymer such as polystyrene. Polystyrene has a permeability high enough that gas exchange with the external atmosphere will significantly change the composition of gas in the small gas spaces over the culture period. The housing 212 may be formed from a variety of materials, a preferred example being polystyrene. In preferred embodiments a barrier layer of a polymer with low permeability, such as PETG, is provided on the base of the housing. Alternatively a secondary container 240 formed e.g. from PETG can be used. The thicker section stopper means 214 is less of a gas permeation path if formed from polystyrene, and can be extended to substantially cover the insert means 216 to reduce gas diffusion through the bulk insert material.

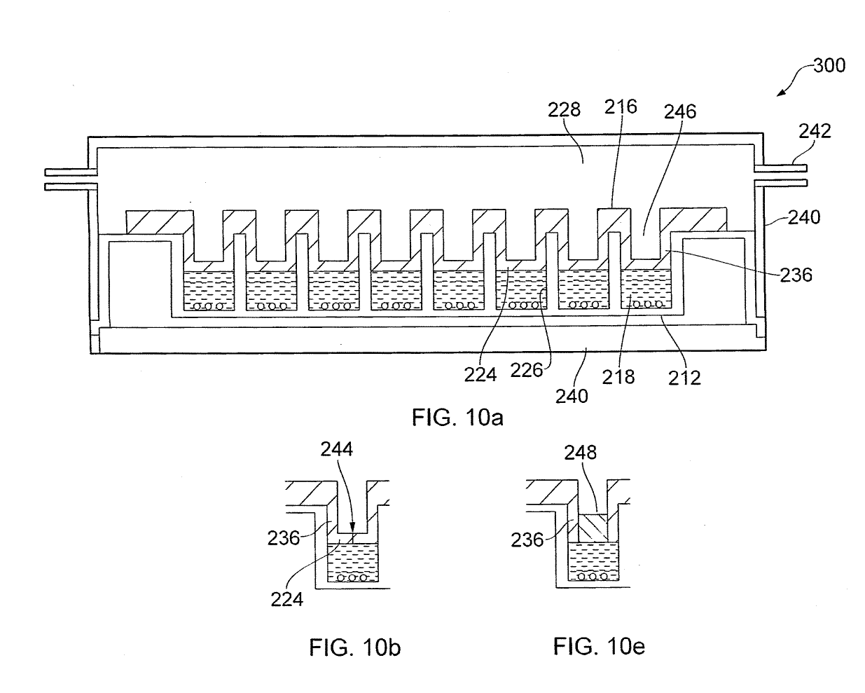

FIG. 10a shows a further embodiment adapted to hold embryos either singly or in small groups in a small volume of media in separate media spaces, so allowing their identity to be tracked. Container 300 comprises a housing 212 as before, the housing comprising one or more wells 226, for example in microtitre plate format. In a preferred embodiment housing 212 is a conventional microtitre plate or microtitre strip, or assembly of strips, of appropriate dimensions (number of wells, well size and shape) to hold the desired number of embryos 220 in the chosen volume of media. The container further comprises an insert 216 provided with one or more well-closing projections 236 each adapted to fit closely a well in the housing 212, defining within one or more of the wells of the housing a media space 218. In preferred embodiments each well 226 of the housing 212 is fitted by a projection 236, leading to a multiplicity of closable media spaces. The insert 216 is similar to flexible elastomeric microtitre plate closures as known in the art, but differs in that it is designed to fit more deeply into the wells and to have known and high gas transport capability into the wells. The media spaces are in diffusive communication with the common gas space 228, preferably by means of a gas channel 246 formed in each of the projections. In preferred embodiments the insert 216 is formed from a gas-permeable elastomeric material such as PDMS, with gas permeability characteristics adequate to allow transport of oxygen across the membrane 224 that closes the well, sufficient to sustain the chosen number of embryos in the well. The gas space 228 is closed by a secondary container 240, comprising a cover and optionally a base. The base may be omitted in some embodiments, for example if a substantially gas-tight seal is formed between the cover and the housing 212, and the base of the wells in housing 212 has low gas permeability—for example if housing 212 is a glass-bottomed microtitre plate.

In use, embryos and media are dispensed into the wells 226 and the insert 216 is fitted. The insert 216 may be formed from a flexible elastomeric material so that it can be inserted into each row of wells in the housing in turn. An insertion aid can be used, such as a roller as known in present technology for fitting flexible microtitre plate closures, or a tool with multiple projections that fit into the gas channels 246 can be used to press the projections 236 into the wells. The insert projections fit into the wells so as to exclude the majority or substantially all of the air initially in the well above the media, and to this end the degree of flexibility of the projections is chosen to allow the air to be displaced. Residual air bubbles in the media spaces 218 rapidly equilibrate with gas from the gas space 228 and so in general are not disadvantageous in this application. The gas space 228 is filled with a controlled gas atmosphere in any convenient way, for example being flushed with gas through optional inlet and outlet ports 242, or being provided with a porous gas-containing body (not shown) as analogous in function to component 122 in FIG. 8, which can be pre-gassed with the desired composition.

FIG. 10b shows a modification to the insert 216 in which the membrane 224 that closes the media space 218 has a slit 244. The slit 244 is normally closed but can be opened, e.g. by air pressure built up in the well by insertion of the projection 236 into the well, or by insertion of a pipette through it from above once the projection has been fitted. The membrane 224 might be shaped to have a recess on the underside where the slit is located to facilitate opening of the slit. By this means air is expelled easily on fitting and/or the contents of the wells can be accessed individually by means of a pipette or needle to allow removal and/or dispensing of media and embryos, without removing the insert from the well or a group of wells. The membrane 224 might alternatively be adapted to allow puncture by a needle or pipette, and optionally re-closure in the manner of a septum, to achieve the same purpose.

FIG. 10c shows a modification to the insert 216 in which part of the base of the projection 236 is formed from a porous component 248, for example formed from a porous hydrophobic material such as VYON™ as already described, which may be a push-fit into the projection. Such a component allows ready venting of air from the well on insertion of the projection, and good gas transport between the media space and the gas space. In some embodiments the component 248 may be adapted to be removable from the projection, so allowing the contents of the well to be accessed.

Typical dimensions of the container in FIG. 10a are chosen to hold the desired number of embryos in the appropriate volume per embryo of media. In a preferred embodiment the housing 212 is a standard flat-bottomed 96-well microtitre plate, or microtitre strip, or assembly of strips, with wells of diameter around 7 mm. A well will therefore hold 200 μl in a depth of 5.2 mm, appropriate for 20 embryos at 10 μl per embryo or, for 10 embryos, 100 μl in a depth of 2.6 mm. For a housing that is a standard 96-well microtitre plate the gas space required for 20 embryos per well for 72 hr extends around 11 mm above the insert and for 10 embryos, 5.5 mm. The dimensions for the insert are not critical; typically the gas channels 246 may have i.d. in the range 1-5 mm and the membrane 22 a thickness of 1-3 mm.

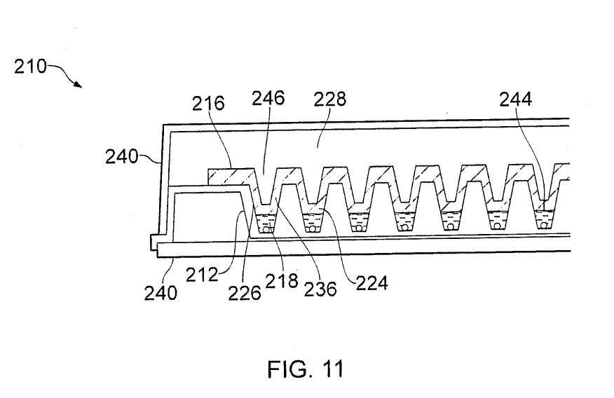

FIG. 11 shows a further embodiment with similar features to those in FIG. 10a shown with the same reference numerals. The housing 212 is now a standard 384-well low volume microtitre plate, for example Greiner Bio-one GmbH, product code 788101, with conical wells that allow a small working volume at the base. The insert 216 is formed from a moulded gas permeable elastomer as before, and is now sized to project into the wells sufficiently far to define a volume suitable for one or a small number of embryos. With typical dimensions of low volume 384-well plates such as the product from Greiner above, a 10 μl volume suitable for culture of a single embryo can be defined. The insert is optionally provided with slits 244 in the media space-sealing membranes 224 as in FIG. 10b. The gas space 228 may be filled with gas in a similar manner to that in the embodiment in FIG. 10a.

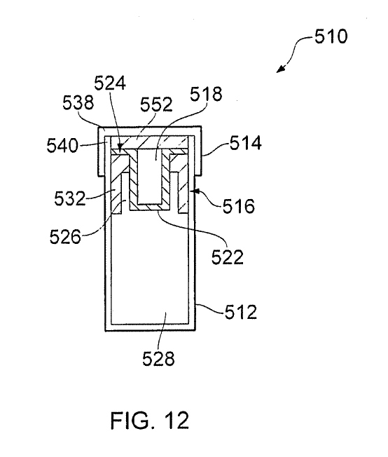

In certain applications it may be desirable for the media to contact a material with conventional, preferably low adsorption and absorption properties, or which can be coated to control cell adhesion properties. Polystyrene and polycarbonate are examples of materials conventionally used in cell biology and embryology which have well-understood surface properties, and oxygen permeability high enough for them to be used in the invention provided wall thicknesses are kept low. FIG. 12 shows a further embodiment in which the container 510 comprises a housing 512, cap 514 and insert 516, the insert now comprising an inner component 522 which has a recess forming the media space 518, mounted in an outer ring 532. The inner component is preferably formed from a rigid polymer such as polystyrene and the outer ring 532 is preferably formed from a resilient material, such as an elastomer, for example PDMS. The inner component preferably has a ring-shaped sealing area 524 surrounding the media space. Preferably one or more gas channels 526 are provided between the inner component and the outer ring, for example by means of a clearance between the inner component and outer ring in the lower portions of the insert.

In use the insert 516 is first fitted part-way into the housing 512 as in previous embodiments, the outer ring being sized to give a friction fit with the housing; when the cap 514 is fitted the inner surface of the cap, or an optional cap insert or seal component 552, bears down onto the seal surface 524, forcing the insert against friction down into the housing and sealing the media space 518. The media and embryos in the media space now contact the material of the inner component and that of the cap or cap seal 552, which can be chosen to be as inert to absorption as is required. In some embodiments the seal region 524 of the inner component may be omitted, the seal then being made against the upper surface of the outer ring. If the seal region 524 is present, then in preferred embodiments it stops short of the inner diameter of the housing, so allowing a portion of the upper surface of the outer ring to be exposed before the cap is fitted. This allows the gas space 528 to be filled with gas of the desired composition by diffusion through the outer ring 532.

In preferred embodiments, the volume of the media space is between 10 μl and 1 ml; in more preferred embodiments between 50 and 500 μl and in most preferred embodiments, between 50 and 200 μl. In an example of the container 510, the housing is formed from a Bibby Sterilin 7 ml bijou vial, the outer ring is moulded from PDMS, e.g. Silastic S (Dow Corning) and the inner component may be formed by for example vacuum forming in polystyrene. The wall thickness of the inner component is preferably between 0.1 mm and 1 mm; more preferably between 0.15 mm and 0.3 mm.

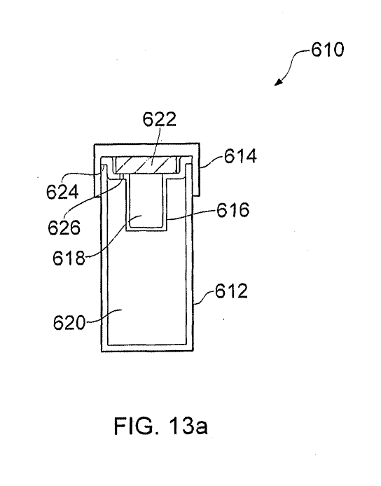

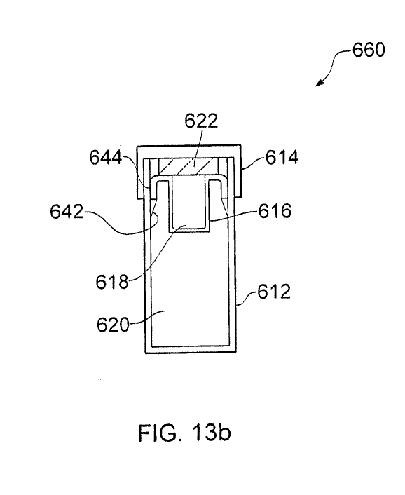

FIG. 13a shows a further embodiment, in which the insert is formed from a moulded thin polymer section and is optionally a separable component, removable from the housing, a separable component that mounts permanently into the housing in use, for example by a snap-fit means, or might be bonded permanently to it. The container 610 comprises a housing 612, cap 614 and insert 616, the insert defining a media space 618 and the insert and housing together defining a gas space 620 as before. In one embodiment the insert 616 is a separate moulded component that rests on the upper rim of the housing at position 624, and when the cap 614 is fitted is brought into firm contact with the rim through force from the cap. Seal component 622 acts to compress the insert evenly around the rim, and is preferably permeable to gas to allow gas supply via the lid as described before. The rim of the housing is preferably smooth and flat in region 624 to allow good sealing. In some embodiments the rim of insert 616 is coated with or enveloped in a region of elastomer, such as PDMS, to assist sealing.

In a preferred embodiment insert 616 is bonded to housing 612 in region 624 with a permanent bond, such as ultrasonic bonding or heat-sealing. In some embodiments the bond is formed so as to be substantially gas-tight.

In preferred embodiments the insert 616 comprises one or more apertures 626, which act to allow a known degree of diffusion of gas between the gas space 620 and an external atmosphere when the cap 614 is absent. This allows ready gassing of the interior of the container when placed in an incubator, and also allows gas communication between the gas space and the seal component 622 in embodiments where this is present, and gas permeable.

The insert may have a stepped cross-section, as in FIG. 13a, which allows the cap to seal against the material of the insert. In an alternative embodiment, the insert is in frictional contact the inside wall of the housing, allowing the cap 614 to bear on the upper rim of the housing, in the same manner as in FIG. 12.

In a further preferred embodiment the insert 616 is supported within the housing by means of features formed on the housing, the insert or both. FIG. 13b shows an embodiment in which the parts are numbered as in FIG. 13a, and the insert 616 is located within the housing 612 by means of one or more features 642 provided on the inner wall of the housing. Optionally the feature 642 is a ridge that acts to support the insert at a position that allows sealing of the media space 618 when the cap is fitted, the insert being a separate component, removable from the housing. Optionally the rim region 644 of the insert extends past the feature 642, and feature 642 interlocks with one or more features (not shown) formed on the rim region 644 so as hold the insert in place within the housing. The container may then be supplied to the user either assembled, with the insert mounted in place, or as two parts, with the insert mounted within the housing by the user before use.

In a further alternative embodiment, the housing and the rim region 644 are so dimensioned that the rim region extends to the base of the housing, so supporting the insert at the correct position; alternatively a further component, such as a cylindrical insert, may be positioned in the base of the housing so as to support the rim region 644 in the same way as shown for the features 642.

In a preferred embodiment the housing may be formed from polystyrene, and the insert 616 may be formed by moulding or vacuum forming, again in polystyrene. Other polymers may be used as appropriate for the required dimensions. The insert wall surrounding the media space is preferably thin to allow adequate gas permeation across it. In preferred embodiments the wall is less than about 0.5 mm thick, in more preferred embodiments less than 0.3 mm and in most preferred embodiments between 0.2 and 0.3 mm thick. The region of the insert against which the seal 622 bears is thick enough to give rigidity to support the sealing pressure, and may be equipped with strengthening ribs (not shown in FIGS. 13a and 13b) to assist this.

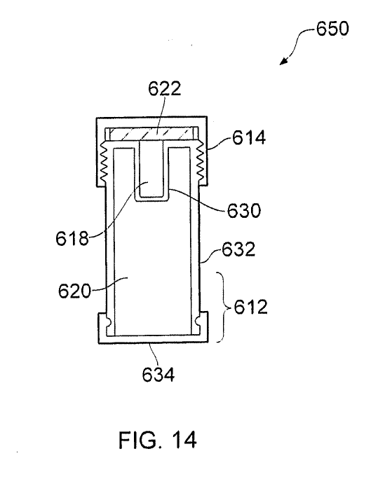

FIG. 14 shows a further embodiment, in which the insert region 630 now forms a permanent part of the housing. The container comprises a housing 612, which is formed from two components: a housing body 632 and housing end cap 634, and a cap 614. The insert region 630 is shaped similarly to that in the embodiments in FIGS. 12 and 13a, but is now either moulded as a single piece with the housing body 632 (i.e. integrally with the housing), or bonded permanently to it. The insert region 630 defines within it a media space 618, closed by the cap 614, and together with the housing 612 a gas space. The cap 614 is preferably a screw fit cap with external threads as shown, though other types of closure are applicable. The housing end cap 634 is shown as a snap-fit cap, though might be a screw-fit cap, or a snap-in plug or any other type of closure that is substantially gas-tight.

In an example of use, the embodiment in FIG. 14 is supplied with the cap 614 and the housing end cap 634 both in place. To gas the gas space, the housing end cap is removed and the container is either filled with a gas stream or left in an incubator—as the open end of the housing is large, the gas equilibration time is advantageously short in this embodiment. The housing end cap is then replaced. The media space if then filled with media plus embryos and the cap 614 replaced. No apertures are shown through the insert region in FIG. 14, though these might be provided to supply gas to the seal component 622.

The container in FIG. 14 is adapted for fabrication by moulding. The housing body 632 is suitable for injection moulding in polystyrene, and the insert region 630 can be made suitably (thin 0.25 mm or less) using this technique. The housing end cap and the lid are standard components that can readily be moulded to fit. The lid seal component 622 is suitably PDMS as before.

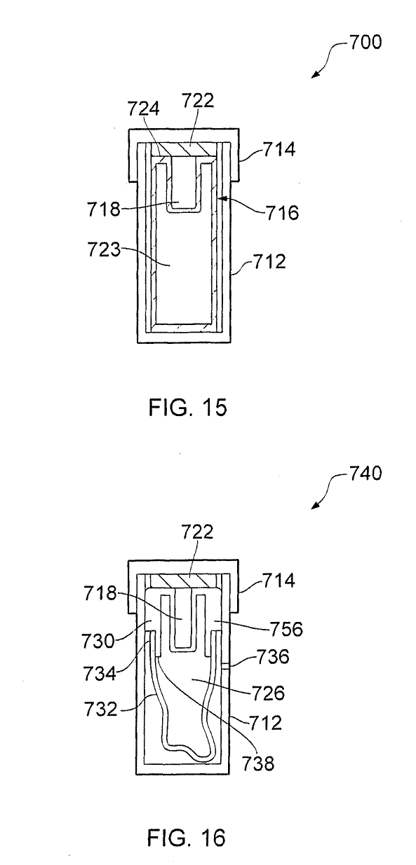

FIG. 15 shows a further embodiment 700 in which the insert 716 comprises a closed gas space 726, the insert mounting inside a housing 712 as before. The closure means 714 forms a liquid-tight seal with the insert to close the media space 718 and a gas-tight seal with the housing 712, to limit inter-diffusion of gas between the gas space 726 and the external environment via the walls of the media space and the media itself. In this embodiment the insert is adapted to have gas-permeable walls around the media space as before, and in preferred embodiments the rest of the insert defining the gas space 726 are substantially impermeable to gas. This embodiment allows the insert 716 to be gassed as before, independently of the housing 712. The insert may be so dimensioned that it forms a loose fit to the inner walls of the housing, while being in contact with the base of the housing when the closure 714 is fitted, so providing reaction force to compress the seal component 722.

Alternatively, the insert may be a frictional fit to the housing as before, that reaction force being provided by friction, or features are provided on the housing, insert or both to provide the reaction force in the manner of FIG. 13b. As in previous embodiments the insert may be provided with one or more apertures (not shown) in the seal region 724, so allowing known gassing characteristics for the gas space.

FIG. 16 shows a further embodiment 740 in which the insert 756 comprises a gas space 726. In this embodiment the insert 756 comprises a more rigid region 730 and a flexible region 732, together defining the gas space 726. The insert is mounted inside a housing 712 and the container is closed by a closure means 714, which closes the media space 718 with a liquid-tight seal as before. Reaction force to sealing by the sealing component 722 is provided by friction between the more rigid region 730 of the insert against the wall of the housing, or by force from features formed on the housing and/or insert as before. This embodiment has the capacity for the gas contained within the gas space 726 to expand in response to lowered external pressure, for example when being transported by air, where cargo holds experience pressures significantly lower than normal seal level atmospheric pressure. A vent 736 may be provided to control or prevent differential pressure across the seal between the housing 712 and the closure 714. As the external pressure drops, the flexible region of the insert expands and in certain embodiments, depending on the material chosen for this region, may stretch, so accommodating the increased volume of gas in the gas space. This has the advantage of substantially avoiding stress on the closure means, so allowing a wide choice of types of housing and closures.

The insert 756 in this embodiment may be formed in a number of ways. In a preferred embodiment the region 730 is formed from moulded polystrene, and is supported at a depth within the housing by features moulded in the housing and/or the region 730. In FIG. 16 the flexible region 732 is shown as a closed tube, sealed to the lower circumference of the more rigid region 730. In a preferred embodiment the flexible region 732 is formed from a bag comprising two sheets of low gas-permeability film, for example metallised mylar, heat sealed together. A hole is formed in one sheet and the end 738 of the more rigid region 730 sealed around the hole, forming an expandable closed space. In use the insert is gassed with the chosen composition, for example by diffusion through the walls of the media space, at atmospheric pressure. The bag forming the flexible region is partly collapsed at this point. As external pressure decreases, the gas inside the insert expands and fully extends the bag. As the pressure falls again, the bag once more collapses. In this construction the material of the flexible region 732 does not have to be stretchable—this is advantageous as such materials often have high gas permeabilities.

The present invention has application also in culture of an extended multicellular structure having a high density of cells per unit area, such as a tissue sample, a skin sample, an organ or part thereof, a cellular layer on a support membrane, or a product in the field of regenerative medicine such as replacement tissue, skin, corneal tissue etc., where availability of oxygen is an important requirement while keeping the concentration of CO₂ dissolved in the media, and hence the pH, within acceptable limits. Conventional culture systems known in the art, such as petri dishes, well plates and the like rely for oxygen availability mainly on diffusion from a gas/media interface, through a depth of media to the cellular structure, and in particular often only facilitate oxygen availability from one side of the cellular structure: for example, in the case of a culture dish formed from low or moderate gas permeability material, to the side closer to the atmosphere above the media. Just as for embryos, oocytes or other cellular structures as previously disclosed, culture of an extended cell layer or tissue sample can be improved by facilitating supply of oxygen to more than one side of the culture space, and in particular to the two sides parallel to the major surfaces of the extended cellular structure. Transport of such cellular structures is also not facilitated by presently available apparatus, as it is not adapted for transport while providing ready oxygen supply. In the situation where transport is done in a container wholly or mainly filled with media, the low rate of diffusion of oxygen through the media may lead to disadvantageously low oxygen concentration in the vicinity of the cellular structure; if the container is only partially filled with media there is a risk that if the container is turned upside down during transport part of the cellular structure will be left without media coverage.

An apparatus for culture of an extended cellular structure in media with oxygen access from both sides of a body of media is disclosed in application US2008/0092027. However, that apparatus is a simple modification of existing culture plate formats; it is unsuitable for operation outside a conventional incubator and makes no provision for operation except at standard horizontal orientation, and hence is unsuitable for culture during transport. Also the geometry and design of the apparatus disclosed does not control, or aim to reduce, the diffusion distance of oxygen through the media, and hence optimise the access of oxygen to the cellular structure.

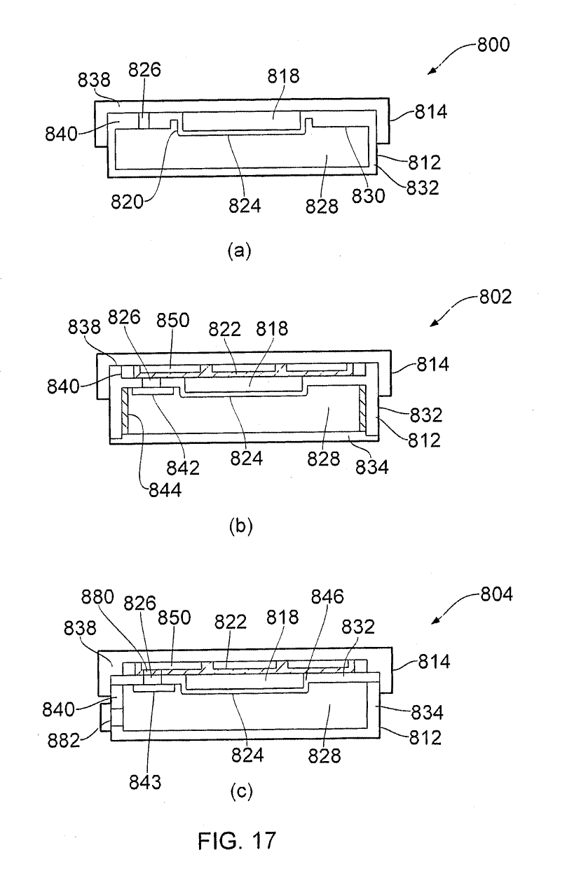

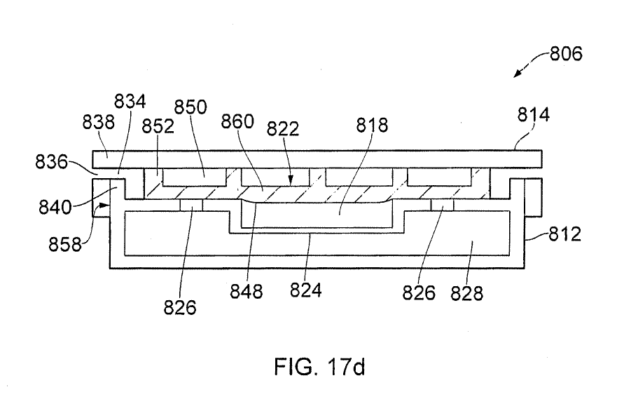

FIG. 17a shows a further embodiment 800 of the invention, in which the media space 818 has a flat aspect ratio and in some embodiments is adapted to retain and locate an extended cellular structure such as those listed above. The container comprises a housing 812, a closure 814 that engages with the housing, a media space 818 and a gas space 828. The closure 814 is adapted to form an essentially gas-tight seal at least in the region of areas 838 and 840 when it is fully fitted to the housing, separating the interior of the housing from the external environment, and also closes the media space 818. The housing 812 comprises a region 824 of higher gas permeability per unit area that forms a barrier between the media space 818 and the gas space 828, and a region 830 that is preferably thicker and capable of supporting a gas-tight seal based on pressure on its upper surface. The region 830 will in general be of lower gas permeability per unit area than region 824. The high permeability barrier region 824 preferably forms most or essentially all of the wall of media space 818 facing the gas space 828. In some embodiments the region 824 extends to the sidewalls 820 of the media space. In the embodiment 800 the media space has a flat geometry, smaller parallel to barrier region 824 than perpendicular to it, so allowing improved diffusion of gas from the gas space, through the barrier 824 and media in the media space, to the side of the media space distant from region 824. Optionally a gas port 826 is provided in the region 830 that allows ready gas exchange between the gas space 828 and the external environment when the closure is loosened or removed, so allowing the gas space to be equilibrated rapidly with a desired atmosphere inside the incubator.

The housing 812 has a lower permeability to gas (e.g. O₂, CO₂) in regions 832 separated by the gas seal region 840 from the region 824, in order to contain the desired gas atmosphere in the gas space 82S. This may be achieved by using the same material but with a thicker profile or by using a different material of lower permeability, or by coating or otherwise reducing the permeability of the wall of the housing. The housing may be formed from bonded subcomponents as would be known to those skilled in the art so as to provide higher and lower gas permeabilities in the desired regions.

In FIG. 17b, the embodiment 802 of the invention comprises components as in embodiment 800 in FIG. 17a, with the addition of a seal component 822 that facilitates a liquid-tight seal for the media space 818, and preferably contributes to supply of gas via diffusion through the seal material as in previous embodiments. Open gas pathways through a closure gas space 850 are optionally provided within the body of the seal component 822 to facilitate this. Optionally a gas port 826 is provided, optionally with a filter 842 to seal the gas space from contamination while the closure 814 is loose or removed. The seal component 822 optionally comprises an opening (not shown) which provides a gas pathway from the gas port 826 to the closure gas space 850. A sealing region 838, 840 is provided to give an essentially gas-tight closure of the container when the closure 814 is fully fitted. In this embodiment the housing may be formed from a first subcomponent 832 and a second subcomponent 834, the first subcomponent comprising the seal support regions and the high permeability barrier region 824, and the second subcomponent being a closure that closes the container. The two subcomponents may be formed from differing materials: for example the first subcomponent from a high gas permeability material such as polymethylpentene (TPX) and the second from a lower permeability material such as polystyrene, though any suitable materials may be chosen as known in the art. Bonding may be by any means known in the art, for example, ultrasonic welding, snap-fit or screw-fit of the two components. Optionally a coating or sleeve component 844 may be used to reduce the permeability per unit area of the higher permeability component where this extends significantly beyond the seal region 838, 840.

FIG. 17c shows a further embodiment 804 of the invention with similar components and features to the embodiment in FIG. 17b. Here the first subcomponent 832 has a lesser extent and the second subcomponent 834 a greater extent, so giving the benefit of a larger area of the lower permeability material. Here an opening 880 in the seal component 822 is shown that provides a gas phase pathway from the port 826 to the closure gas space 850.

Optionally, in all embodiments of the invention, a gas closure means may be provided that allows access from the exterior to the gas space, so allowing exchange of gas between a gas environment, for example in a conventional gas incubator, and the gas space while the media space is closed by the closure 814. FIG. 17c shows such a gas closure means 882 provided in the wall of the housing. Examples of such gas closure means are a plug, screw cap, tap or the like. One or more such gas closure means might be provided, optionally opening or closing one or more access regions opening to the gas space. The opening might be such that gas exchange takes place mainly by diffusion or passive convection within an incubator, or might be adapted for active flushing of the gas space, for example with a gas-line connection, optionally with one or more valves controlling the gas pathway through the connection.

The closure 812 may engage with the housing in any manner known in the art, though a fitting that does not substantially pressurise the interior of the container is preferred. Preferred embodiments include a screw fitting with a thread either on the inside or the outside of the housing, i.e. either inside or outside the seal region 838, 840. A snap fitting may also be used. The closure may have a partial closure condition that leaves a gas path open from the exterior to the interior of the housing and a fully closed condition that restricts gas passage between the exterior and the interior. For example in the case that the closure 814 has a screw thread engaging with the housing the gas pathway to the interior might be open when the lid is partially screwed down and closed when it is fully screwed down. One or more location means may be provided on the closure, the housing or both to facilitate location in one or more closure condition. For example, a protrusion and matching recess might be used to provide a ‘click-stop’ action to locate the closure in conditions with the open and the closed gas pathway.

FIG. 17d shows a further embodiment 806 in which the closure 814 is adapted to have a first condition in which it closes the media space 818 while leaving a gas pathway open from the exterior to the interior of the container, and a second condition in which the media space is closed and the gas pathway is also closed. In preferred embodiments the sealing component 822 in the lid is adapted to close the media space while leaving further compression possible as the closure is moved from the first condition towards the second. In FIG. 17d the container 806 is comprises a sealing component 822 mounted in the closure, in turn comprising a closure gas space 850 which provides a gas pathway within the seal component, a seal surface 848 that closes the media space and a second barrier region 860 that allows diffusion of gas between the closure gas space and the media space. Sealing component contact regions 852 are sized to control the spacing of the sealing surface 848 of the seal component from the inner surface of the closure when the seal component 822 is uncompressed. The closure gas space 850 allows ready diffusion of gas from the gas space 828 to the barrier 860, optionally facilitated by a gas port 826 and optionally by an opening (not shown) through the seal component to the gas space 850 that aligns with the gas port 826 as shown in FIG. 17c. The seal component 822 is preferably mounted in the closure 814 for example by adhesion or physical interlocking of regions of the seal component with regions of the closure. Alternatively the seal component might be separable from the closure and in use mounted in the container separately from it.

In this and other embodiments the seal component and the closure may be dimensioned so that the seal surface closes the media space 818 in a first condition of the closure, while leaving a gas pathway open between the interior of the container and the exterior. As shown in FIG. 17d, the seal component may be sized so that as the closure 814 is applied to the housing, a first position is reached where the media space 818 is closed by the seal surface 848 while the gas sealing regions 838 and 840 are not in contact. The sealing component is preferably compliant, for example the sealing component contact regions 852 are preferably deformable, allowing the closure to be moved relative to the housing to facilitate closure of the gas sealing regions 838, 840 while keeping the media space closed by seal face 848. The seal surface 848 might also be profiled to effect closure of the media space, for example with a profile that projects slight into the media space when the media space is closed.

One or more ports 856 through the closure may be provided that assist gas access to the interior of the housing in the open condition. A gas pathway might also be provided via the region 858 between the closure and housing, for example via screw thread, a groove in the surface of the closure or the housing or similar means. The container might be approximately cylindrical and the closure 814 might be a screw fit to the housing; alternatively the closure might be a snap-fit to the housing, preferably with two snap-fit positions, a first position as shown in FIG. 17d with the gas pathway open and a second position in which the closure snap-fits lower down on the housing with the seal regions 838 and 840 in contact. It will be apparent that any of the features described for the embodiment 806 might be applied to other embodiments of the invention.

The embodiments in FIGS. 17a-d may be fabricated by any appropriate means and from a range of appropriate materials as known in the art. Preferably the gas-permeable barrier regions 824, 860 are formed from a material of high gas permeability, such as a high permeability elastomer, for example PDMS, or a high permeability rigid polymer such as polymethylpentene (TPX). Alternatively they may be formed from thin section of a polymer of lower permeability, such as polystyrene or polycarbonate. In the embodiments in FIGS. 17a-d the first barrier region 824 may either be formed as part of the housing 812 by moulding, or might be formed by bonding a separate component onto a component of the housing, for example by forming barrier region 824 from an area of polymer film mounted on or bonded to the housing (for example on or to housing component 832 in FIGS. 17b and 17c) at the circumference of the media space, shown as 846 in FIG. 17c. This avoids the need to mould a thin section in a thicker moulded component. In a preferred example the housing material may be polystyrene and the barrier may be formed from polystyrene film, or more preferably TPX film, and the bond may be formed by ultrasonic bonding. Alternatively the film might be mounted onto the housing or a component of the housing by e.g. a snap-fit mounting collar that fits around the outside of the wall 820 of the media space (see FIG. 17a) so holding the film in place. The housing and closure are preferably formed from a low permeability polymer such as polystyrene or PETG, to reduce the gas of diffusion of gas between the gas space 828 and the exterior through the walls of the container. The polymer film forming the barrier region might be used as supplied in essentially planar form, or might be formed as a specific component for example by moulding, embossing or the like. Supporting structures, such as ribs, might be formed as part of the barrier to increase rigidity.

Any size or shape of the container is within the scope of the invention. In preferred embodiments the dimensions of the container and its features are chosen in the light of the materials chosen for fabrication, taking into account their permeability to gas and the thickness or other dimensions or features that are needed to render them suitable for use in the design, for example to give them mechanical stability. They are also chosen to suit the type of cellular structure to be cultured and/or transported, taking into account aspects such as its size, any support or backing materials such as support membranes associated with the cellular structure, its oxygen demand and its consumption of nutrients dissolved in the media such as glucose. For certain cellular structures such as embryos or oocytes preferred volumes of media per structure are used for culture and so the media space has preferred volumes which depend on the number of cellular structures to be cultured and/or transported together, as previously described. In preferred embodiments for embryos or oocytes the volume of the media space will be in the range 0.1-100 ul per embryo or oocyte to be transported.

Other cellular structures do not require a preferred volume; rather considerations of oxygen access and easy handling are important. Diffusion through the media is usually the limiting factor on oxygen supply to the cellular structure, and in preferred embodiments the diffusion distance through the media is chosen to provide a concentration at the cellular structure that is estimated to be appropriate for the cellular entity to be cultured. In preferred embodiments for extended cellular structures the maximum distance within the media space from the gas/media barrier to a position where the cellular structure might be located (for example if it is free to sediment)—is preferably between 0.01 and 10 mm, more preferably between 0.01 and 5 mm. In preferred embodiments adapted for extended cellular structures the media space is preferably between 0.1 and 10 mm between its major surfaces, more preferably between 0.5 and 5 mm.

Diffusion limitation through the barrier will also limit the oxygen flux to the cellular structure, or result in a lower oxygen concentration at the cellular structure for a given flux. Dimensions of the one or both barriers depend on the permeability of the barrier structure(s) and are chosen so us not to add excessively to the overall diffusional impedance for oxygen between the gas phase and the media. The barrier may have thicker portions for support and thinner portions for gas diffusion. The following preferred dimensions are for the thinner, gas diffusion portions. Depending on the material used, the barrier is preferably between 0.02 and 10 mm thick and more preferably between 0.05 and 5 mm thick. For example, for a barrier formed mainly from a high permeability elastomer such as PDMS the thickness is preferably in the region 0.2 to 5 mm, for a barrier formed mainly from a high permeability rigid polymer such a TPX the thickness is preferably in the region 0.05 to 1 mm, more preferably in the range 0.05 to 0.4 mm, and for a barrier formed mainly from a lower permeability rigid polymer such a polystyrene the thickness is preferably in the range 0.02 to 0.3 mm, more preferably in the range 0.05 to 0.2 mm. It will be apparent to a skilled person that the thickness of the barrier, or other dimensions of the container, can be chosen appropriately with regard to the permeabilities of the chosen materials.

Typical permeabilities P(O₂) for oxygen transport through polymers that might be used for the barrier material are given in units of 10⁻¹³ cm³.cm.cm⁻².Pa⁻¹ as polystyrene (PS): P(O₂)=2; TPX: P(O₂)=20; PDMS P(O₂)=400. [Goodfellow, Inc., materials supply catalogue (www.goodfellow.com) download 18^{th} April 08. P(O₂) is quoted at 25° C. for PS and TPX, at 0° C. for PDMS—for the purpose of order of magnitude estimation in this example the differences between P(O₂) at 25° C., 0° C. and the typical operating temperature of 38° C. are not important].

With these parameters, dimensions of the barrier(s) 824 (860) may be chosen based on a known or estimated oxygen flux per unit area to the cellular structure, using Fick's law of diffusion to give the drop in concentration across the barrier(s) as will be apparent to those skilled in the art. For illustration, examples of measurements of oxygen uptake rates (OUR) for different cell types are given in Peng C-A, Paulson B. O., Annals Biomedical Engineering 1996 vol. 24(3) p. 373-381, Cho et al., Biotechnology and Bioengineering 97(1) 2007 p. 188-199, and discussion of oxygen diffusion in media in Mentzen et al. Respiration Physiology 100 (1995) p. 101-106. For example, using OUR figures from these references, for a cellular structure with 10⁶ cells.cm⁻², a barrier formed from PS has a preferred thickness range is 0.02-0.1 mm; a TPX barrier has a preferred thickness range of 0.02-0.4 mm and a PDMS barrier has a preferred thickness up to 5 mm. For cellular structures of different OUR per cell and/or different cell densities per unit area, the preferred dimensions may differ from the above.

The container of the invention preferably provides a ready gas diffusion pathway from the gas space to the side of the media space remote from the first barrier. In preferred embodiments this is provided by a gas phase diffusion pathway through a closure gas space 850 and a second barrier 860 (FIG. 17d). The diffusion constant of oxygen is such much grater than in solid or liquid materials that a gas-phase diffusion pathway offers negligible additional resistance to oxygen transport. The closure gas space (54 in FIG. 2, 850 in FIG. 17d) is supplied with gas from the gas space 28, 828, by diffusion through an optional gas port (826) in the housing and then through the material of the seal component in some embodiments and optionally through a port (880, FIG. 17c) leading through the seal component to the closure gas space. The above calculation shows that a preferred distance through of PDMS in the gas transport pathway is in the range 0-10 mm per square cm area of the pathway.

The gas space 828 is dimensioned to contain enough of the desired atmosphere to supply oxygen to the cellular structure and to compensate for losses to the exterior through gas permeable components or through the gas closure seal. In preferred embodiments the gas space has a volume in the range 0.1-100 ml, in more preferred embodiments in the range 1-40 ml.

It will be evident to the skilled person that based on the above discussion the container according to the invention can be designed and sized to suit a wide range of cellular structures of different oxygen demand.

### EXAMPLES

### Example 1

A container as in FIG. 1a was designed to culture 50 embryos in a 5% oxygen, 5% carbon dioxide atmosphere for 72 hr. The container used as housing 12 a 7 ml capacity polystyrene Bijou vial (Bibby Sterilin, product code 129A) with an externally threaded screw cap. The insert 16 was moulded in Silastic S PDMS (Dow Corning) mixed in the standard ratio according to the supplier's instructions. The media space 18 was of diameter 7 mm, height 13 mm to give a volume of 500 μl, suitable for 50 embryos at 10 μl media per embryo. The walls 22 and base 24 were 1.5 mm thick and the gas channels 26 were 1.5 mm across in the radial direction, extended to 1 mm short of the surface 36 and occupied 50% of the circumference on which they were situated.

The insert was sized to be an interference fit with the wall of the vial and on filling the media space with 500 μl media, fitting the cap forced the insert down into the vial and provided leak free sealing of the media space.

### Total Oxygen Demand

The total oxygen demand for 50 embryos over 72 hr is 1.8E-7 mol (assuming an individual oxygen demand of 1.4E-14 mol.s-1 per embryo (H. Shiku et al., Anal. Chem. (2001) 73(15) 3751-8), equivalent to 0.87 ml of gas (at 25 C) if the gas is depleted from 5% to 4.5% oxygen. The gas space 28 was approximately 5.3 cm3 and so oxygen depletion in the gas space (neglecting other factors and loss through the lid) is approximately 0.1%.

### Leakage Through the Walls of the Gas Space

The change in gas composition owing to diffusion through the walls of the vial is exponential with a time constant that depends on the volume and surface area of the gas space, the wall thickness and gas permeability of the vial. Polystyrene Bibby Sterilin bijou vials have a quoted carbon dioxide permeability of 75E-10 mm.cm3.cm-2.(cm Hg)-1.s-1, and oxygen permeability of 15E-10 mm.cm3.cm-2.(cm Hg)-1.s-1 (www.barloworld.com, Sterilin website, download March 2007). For a gas space of volume 5.3 cm3 and a wall thickness of 1.5 mm (measured) the time constant for loss of gas through the wall (at the same total pressure inside and out) is 275 hr. For an initial internal atmosphere of 5% carbon dioxide and air outside, this leads to a change from 5% to 4% carbon dioxide in approximately 61 hr, and 5% to 3.5% in 98 hr. This is small enough not to compromise embryo development through change in pH of the media. If the internal oxygen content is 5%, oxygen will diffuse inwards from an air atmosphere: the diffusion coefficient is 5 times lower than that of carbon dioxide and the driving force (20% vs. 5%) is 3 times higher—so the increase in percent oxygen will not be significant over 72 hr.

### Oxygen Flux to the Embryos

Diffusion of oxygen to the embryos was estimated (i) with the embryos resting on the base 24 of the media space and (ii) with them resting on the lid. Having the embryos resting on the base is the best case and, given the provision of the gas channels 26 and the thin wall in their vicinity, is also a reasonable estimate of the situation when the container is on its side. Having the embryos resting on the lid is the worst case, in that the lid is assumed to be impermeable and O2 diffusion to the embryos is assumed to be wholly through the media.

### (i) Embryos on the Base or Walls

Oxygen arriving at the embryos through the PDMS is in excess of that needed for respiration of a group of 50 embryos arranged in a disc at an embryo:gap ratio of 1:1 in hexagonal symmetry. The additional contribution from hemispherical diffusion is calculated below. Oxygen solubility in PDMS=0.18 cm3(STP)/cm3.atm and diffusion coefficient 3.4E-5 cm2.s-1 (Merkel et al. (1996) quoted in Zanzotto et al., Biotechnology and Bioengineering 87(2) (2004) 243-254). For 50 embryos in a disc at an embryo:gap ratio of 1:1 in hexagonal symmetry, the disc radius is 0.085 cm, and the oxygen flux for non-diffusion limited respiration (=50×1.4E-14 mol.s-1) creates a concentration at the inner PDMS surface of 3.2E-8 mol.cm-3, equivalent to that in equilibrium with gas containing 3.2% oxygen. The distance in the media from the wall to the embryos is assumed to be negligible (e.g. 10 um) and so contributes negligibly to the overall concentration gradient between the gas atmosphere and the embryos. Oxygen concentration of 3.2% is sufficient for good bovine embryo development (J. G. Thompson et al. J. Reproduction and Fertility 118 (2000) 47-55). If 20% oxygen is used in the gas atmosphere, clearly even less of a diffusion limitation problem will arise.

### (ii) Embryos Resting on the Lid

For embryos resting on the lid, the concentration at the embryos is found using the hemispherical diffusional impedance from the nearest gas phase source, through the media to the embryos. The embryos are assumed to be arranged in hexagonal symmetry with a gap:embryo ratio ranging from 0 (the embryos are in contact in hexagonal close packing) to 3. Obviously the embryos will be arranged in a more random way, though the spacing may be comparable. For comparison in the example dimensions above uniform distribution of the embryos over the lid or the base of the media space is at gap:embryo ratio of only 7 (for media space diameter=7 mm and embryo diameter=100 um), and if the embryos or oocytes tend to group together for example by sedimentation to a confined lowest part of the container if this is tilted, or because the embryos or oocytes are sticky and tend to stick together if they touch, some or all of the embryos could become closely packed in practice. For ease of estimation the properties of PDMS are taken the same as those of media (water). This is justified as (a) the PDMS containment of the media space has relatively thin walls (1.5 mm) compared with the typical diffusion dimension in the media (which is a minimum at approximately the radius of the media space, 3.5 mm, and a maximum at approximately the length of the media space, 13 mm), and (b) in the hemispherical diffusion geometry the outer dimension of the diffusion region and the properties of the region near it have much less impact on the diffusional impedance than does the radius of the inner boundary of the diffusion region and the properties of the region near the inner boundary.

An oxygen concentration at the embryos of 1E-8 mol.cm3, the concentration in the media in equilibrium with 1% oxygen in the gas phase, is considered to be the minimum desirable for bovine embryo culture (Thompson et al. (2000) op. cit.). The flux to the embryos is then calculated using hemispherical diffusion in elements of the cylindrical media space integrated in spherical coordinates over the cylinder.

The critical parameter in the estimation is the inner radius of the diffusion region, which is taken here to be a hemisphere of radius a=(2/pi) r(disc), where r(disc) is the radius of the disc over which the embryos are arranged as described above, by analogy to the result for diffusion-limited current at disc microelectrodes (K. B. Oldham and C. G. Zoski, J. Electroanal. Chem. 256 (1988) 11-19). The results show that for a separation between embryos of less than 3 diameters, respiration will be diffusion limited with local concentration equivalent to equilibrium with 1% O₂ or below, even for optimally small diffusion distances from a gas-phase source (that cannot be realised in a practical design). It is unlikely that embryos will group together closely by chance (though if they show tendency to stick together, or to the wall of the media space, such that random movement in the media will lead to aggregation, that assumption might break down). Therefore an embryo gap:space ratio of 3 was used, with resulting r(disc)=0.165 cm and a=0.105 cm.

The flux to the group of 50 embryos in the embodiment in FIG. 1a was calculated as approx. 6E-13 mol.s-1, compared with a non-diffusion-limited oxygen requirement of 7E-13 mol.s-1. Given the uncertainty of the estimation, the probability that the embryos would be arranged in a more favourable way, and the resulting equivalent gas-phase oxygen concentration of 0.9% O2 that that flux would imply, this was considered to be confirmation that the design would be adequate in most circumstances. Oxygen supply can be increased by including a gas diffusion path in the lid, as shown in FIG. 2 and example 2 below.

The largest contribution comes from the shortest diffusion path, radially from the wall of the media space closest to the embryos; this shows the importance of the gas channels 26 in providing ready gas access to this region. Without these channels, the oxygen diffusion rate to the embryos when resting on the lid or the walls 22 would be much lower.

Time to Equilibrate the Gas in the Gas Space with an External Gas Atmosphere

The main route to equilibration is diffusion through the insert, in particular diffusion through the wall 22 and base 24 of the media space, and the composition will change exponentially with a time constant that depends on the volume of the gas space, the permeability and effective area and thickness of the insert and any pressure differential across the insert (which will be negligible in practice as gas tends to leak past the insert as it is being pushed into the housing—it will be zero if the insert has a gas through path 42 as in FIG. 1c). Gas will also diffuse through the wall of the vial if that is formed from a polymer with an appreciable permeability, but at a much lower rate. For the PDMS parameters listed above, the container in example 1 has a time constant for oxygen equilibration with the outside atmosphere of approx. 3 hr, so for 99% equilibration (i.e. an initially air filled interior will reach 5.15% oxygen) the container should be left in the desired gas atmosphere for approximately 13 hr. The diffusion coefficient of carbon dioxide in PDMS=2.0E-5 cm2.s-1 is lower than that of oxygen, so giving a time constant for carbon dioxide equilibration of approx. 5 hr and 99% equilibration time (for 4.95% carbon dioxide in the gas space in a 5% carbon dioxide external atmosphere) of 22 hr.

The corollary of this is that if the container is removed from the controlled gas atmosphere and left uncapped while embryos are loaded the gas concentration inside the vial will change with the same time constants: the carbon dioxide concentration in the gas space will fall from 5% to 4.5% in around 30 minutes, and the oxygen concentration will rise from 5% to 6% in 11 minutes, which gives adequate time for the media to be added and the cap closed before excessive shift in concentration.

### Example 2

A container as in FIG. 2 was designed to culture 50 embryos in a 5% O2, 5% CO2 atmosphere for 72 hr.

The lid component 52 was designed to augment dissolved oxygen availability at a group of embryos resting on the lid when the container is upside down. Availability of sufficient flux of dissolved oxygen at a group at the centre of the lid means that at least this amount will be available at a position towards the edge of the lid, for example when the container is upside down and tilted away from vertical.

The flux contribution through the PDMS lid is modelled by calculating the sum of diffusional impedances (i) through the annulus represented by the PDMS in region 58 in FIG. 2, (ii) radially through the bulk of the PDMS lid component to an inner radius equal to the thickness of the lid component and (iii) hemispherical diffusion from the edge of the cylinder bounded by that inner radius to the disc on which the embryos rest. In the example the gas channels are taken to occupy 50% of the annular area 58, and the bridges (30 in FIG. 1b) 50%; the region 58 is 2 mm thick, and the embryos are taken to be arranged with a gap:embryo ratio of 3:1, i.e. on a disc of radius 1.65 mm as before. For the O2 diffusion and solubility parameters as above, with a local concentration at the embryos equivalent to that in media in equilibrium with a 1% oxygen atmosphere, the flux to the embryos is approx. 3E-12 mol.s-1—much greater than the non-diffusion-limited respiratory flux of 50×1.4E-14=7E-13 mol.s-1. For a flux of 7E-13 mol.s-1, the concentration in the media at the embryos is 4.2E-8 mol.cm-3, equivalent to that in media in equilibrium with a 4.3% oxygen atmosphere.

This shows that a solid PDMS lid component 52 provides sufficient O2 diffusional flux to maintain a group of 50 embryos at 3:1 spacing free of respiratory limitation through O2 diffusion limitation. The lid component 52 might also be provided with a gas space 54, which would serve to increase the diffusion rate if a larger number of embryos (or lower O2 content atmosphere) were to be used.

### Example 3

The gas channels 26 are closed by the rim 64 and so diffusion via this route, while non-zero, will be negligible compared with through a vent channel 66. The channel 66 in FIG. 3c extends the length of the insert 16 in order to present the maximum resistance to diffusion. Change of composition of the gas in the gas space is an exponential process as described above, with time constant tau=V.1/(D.A), where V is the volume of the gas space, D is the diffusion coefficient of the gas of interest (carbon dioxide here, D=0.16 cm2.s-1 in air at 20 C), 1 and A are the length and areas of the substantially rectangular cross-section channel. For typical dimensions: V=5.3 cm2; A=0.01 cm2 and 1=1 cm, tau=55 minutes and the time for a fall in carbon dioxide concentration in the gas space from 5% to 4.8% is around 2 minutes, which is an adequate performance for the design. If the time is required to be longer, the channel area can be smaller or the volume V larger.

1. A container for culturing and/or transporting embryos, oocytes or other cellular structures, comprising a housing (12) having a gas space (28) and a media space (18) for a liquid medium separated from one another by a barrier (16) having one or more gas permeable regions to allow gas diffusion from the gas space to the media space, and an essentially gas-tight gas closure means (14) adapted to restrict the passage of gas into the container from the exterior environment, characterized in that the container further comprises liquid closure means (14, 16) adapted to engage with the housing to form a liquid-tight seal for retaining liquid in the media space.

2. A container as claimed in claim 1 in which the barrier (16) forms at least part of an insert mounted in the housing.

3. A container as claimed in claim 1 in which the housing and the barrier are formed in one piece.

4. A container as claimed in any preceding claim in which the barrier is provided with one or more channels or indentations (26) to increase the diffusion of gas from the gas space to the media space.

5. A container as claimed in claim 2 in which the insert defines a vent channel (42) to allow gas to escape from the gas space when the insert is being inserted into the housing.

6. A container as claimed in any preceding claim in which the gas closure means is releasably securable to the housing.

7. A container as claimed in any preceding claim in which the barrier is at least partly porous, and the pores in the barrier comprise at least part of the gas space.

8. A container as claimed in any preceding claim in which the gas closure means together with the barrier defines the media space.

9. A container as claimed in any preceding claim in which the gas closure means together with the barrier defines the gas space.

10. A container as claimed in any preceding claim in which the gas space comprises a flexible region adapted to allow increase in volume of the gas within the gas space.

11. A container as claimed in claim 2 in which the insert encloses the gas space.

12. A container as claimed in claim 2 in which the insert is resilient.

13. A container as claimed in claim 2 in which the insert comprises a rigid polymer body attached to a resilient outer sleeve.

14. A container as claimed in any preceding claim in which the barrier includes a punctureable membrane which allows pipetting of liquid media through the barrier.

15. A container as claimed in any preceding claim in which the barrier includes a membrane having a slit which forms a vent channel or which allows pipetting of liquid media through the barrier without removing the barrier.

16. A container as claimed in any preceding claim in which the barrier includes a removeable porous region.

17. A container as claimed in any preceding claim in which the liquid closure means is provided with a gas permeable layer which is configured to close the media space to increase gas diffusion from the gas space to the media space.

18. A container as claimed in any preceding claim in which the liquid closure means includes a gas space in gas communication with the gas space and the media space.

19. A container as claimed in any preceding claim in which the barrier defines a plurality of liquid media spaces having a common gas space.

20. A container as claimed in any one of claims 1 to 18 in which the barrier defines a plurality of liquid media spaces having respective gas spaces.

21. An insert for use in a container as claimed in claim 2.

22. An apparatus for transporting embryos, oocytes or other cellular structures, comprising: a container as claimed in any one of claims 1 to 20, and a transportable incubator.

23. A method for culturing and/or transporting embryos, oocytes or other cellular structures (collectively, ‘objects’) comprising the steps of:
a. Providing a container comprising a housing (12) having a gas space (28) and a media space (18) for a liquid medium separated from one another by a barrier (16) having one or more gas permeable regions to allow gas diffusion from the gas space to the media space, and an essentially gas-tight gas closure means (14) adapted to restrict the passage of gas into the container from the exterior environment, and liquid closure means (14, 16) adapted to engage with the housing to form a liquid-tight seal for retaining liquid in the media space;
b. Establishing a desired gas composition within the gas space;
c. Filling the media space with media plus one or more objects
d. Fitting the gas closure means and the liquid closure means to close the media space and to close the gas space from communication with the environment.

24. A method for culturing and/or transporting embryos, oocytes or other cellular structures (collectively, ‘objects’) comprising the steps of:
a. Providing a container set of parts comprising a housing, a closure means and an insert, the container set of parts when assembled comprising a gas space and a media space both closed from the external environment;
b. Filling the media space with media plus one or more objects
c. Fitting the insert together with the housing to create a substantially liquid tight seal between an outer face of the insert and an inner wall of the housing, so closing the media space;
d. Establishing a desired gas composition within the gas space; and
e. Fitting the closure means to close both the media space and gas space from communication with the environment.

25. A method as claimed in claim 23 or 24 in which the desired gas composition is provided by diffusing a suitable gas into a porous insert prior to assembly.

26. A method as claimed in claims 23 to 25 further comprising: incubating the assembled container and one or more objects.
